(19) United States
(12) Patent Application Publication
(10) Pub. No.: US 2023/0045873 A1
(43) Pub. Date: Feb. 16, 2023

(54) ANTI-oxMIF/ANTI-CD3 BISPECIFIC ANTIBODY CONSTRUCTS

(71) Applicant: OncoOne Research & Development GmbH, Klostemeuburg (AT)

(72) Inventors: Alexander SCHINAGL, Vienna (AT); Michael Robert THIELE, Seyring (AT); Randolf KERSCHBAUMER, Klosterneuburg (AT)

(21) Appl. No.: 17/782,903

(22) PCT Filed: Dec. 4, 2020
(86) PCT No.: PCT/EP2020/084676

§ 371 Date: Jun. 6, 2022

(30) Foreign Application Priority Data
Dec. 6, 2019 (EP) 19214037.4

(51) Int. Cl. C07K 16/28; C07K 16/24; A61P 35/00; C12N 15/63

(52) U.S. Cl. CPC C07K 16/2809; C07K 16/24; A61P 35/00; C12N 15/63; C07K 2317/622; C07K 2317/55; C07K 2317/31; C07K 2317/565

(57) ABSTRACT

The invention refers to an anti-oxMIF/anti-CD3 antibody comprising at least one binding site specifically recognizing oxMIF and one binding site specifically recognizing CD3, which is an IgG wherein a scFv is fused to only one of the two heavy IgG chains, an IgG wherein one Fab arm is replaced by a bispecific-T-cell-engager (BiTE), or an IgG wherein both Fab arms are replaced by scFvs with different binding specificities, and its use in the treatment of hyperproliferative diseases, specifically in the treatment of cancer.

### FIELD OF THE INVENTION

The invention refers to an anti-oxMIF/anti-CD3 antibody comprising at least one binding site specifically recognizing oxMIF and one binding site specifically recognizing CD3, which is an IgG wherein a scFv is fused to only one of the two heavy IgG chains, an IgG wherein one Fab arm is replaced by a bispecific-T-cell-engager (BiTE), or an IgG wherein both Fab arms are replaced by scFvs with different binding specificities, and its use in the treatment of hyperproliferative diseases, specifically in the treatment of cancer.

### BACKGROUND

The cytokine Macrophage Migration Inhibitory Factor (MIF) has been described as early as 1966 (David, J. R., 1966, Proc. Natl. Acad. Sci. U.S.A. 56, 72-77; Bloom B. R. and Bennet, B., 1966, Science 153, 80-82). MIF, however, is markedly different from other cytokines and chemokines because it is constitutively expressed, stored in the cytoplasm and present in the circulation of healthy subjects. Due to the ubiquitous nature of this protein, MIF can be considered as an inappropriate target for therapeutic intervention. However, MIF occurs in two immunologically distinct conformational isoforms, termed reduced MIF (redMIF) and oxidized MIF (oxMIF) (Thiele M. et al., J Immunol 2015; 195:2343-2352). RedMIF was found to be the abundantly expressed isoform of MIF that can be found in the cytoplasm and in the circulation of any subject. RedMIF seems to represent a latent non-active storage form (Schinagl. A. et al., Biochemistry. 2018 Mar. 6; 57(9):1523-1532).

In contrast, oxMIF seems to be the physiologic relevant and disease related isoform which can be detected in tumor tissue, specifically in tumor tissue from patients with colorectal, pancreatic, ovarian and lung cancer (Schinagl. A. et al., Oncotarget. 2016 Nov. 8; 7(45):73486-73496).

The number of successful drug targets to treat cancers, like the above mentioned oxMIF positive indications, is restricted. E.g. more than 300 potential immune-oncology targets are described, but many clinical studies focus on anti-PD1 and anti-PDL1 antibodies (Tang J., et al. Ann Oncol. 2018 Jan. 1; 29(1):84-91). The scientific and medical community therefore eagerly awaits potential drugs targeting tumor specific antigens to increase the therapeutic options for cancer patients with poor prognosis.

OxMIF seems to be highly tumor specific, and antibodies targeting oxMIF show efficacy in vitro and in animal studies (Hussain F. et al., Mol Cancer Ther. 2013 July; 12(7):1223-34, Schinagl. A. et al., Oncotarget. 2016 Nov. 8; 7(45):73486-73496). An oxMIF specific antibody demonstrated an acceptable safety profile, satisfactory tissue penetration and indications for anti-tumor activity in a phase 1 clinical trial (Mahalingam D. et al., 2015, ASCO Abstract ID2518, Mahalingam D. et al. 2020, Br J Clin Pharmacol, 86(9), 1836-1848). However, the mode of action of anti-oxMIF antibodies seems to be solely based on neutralization of the biologic activity of oxMIF. The antibodies did not show any bystander effect such as complement-dependent cellular toxicity (CDC) or antibody-dependent cellular cytotoxicity (ADCC) (Hussain F. et al., Mol Cancer Ther. 2013 July; 12(7):1223-34).

Del Bano J. et al. provide a general review on bispecific antibodies for use in cancer immunotherapy (ANTIBODIES, vol. 5, no. 1, 2015, page 1).

In WO 2009/086920 A1 anti-MIF antibodies are described

WO 2016/156489 A1 refers to a dosage regimen of anti-MIF antibodies.

WO 2016/184886 A1 describes anti-MIF antibodies in the treatment of tumors containing mutant TP53 and mutant RAS.

KERSCHBAUMER R. J. et al. report neutralization of Macrophage Migration Inhibitory Factor (MIF) by fully human antibodies (JOURNAL OF BIOLOGICAL CHEMISTRY, vol. 287, no. 10, 2012, pages 7446-7455).

Douillard P. et al. disclose human antibodies specific for oxidized macrophage migration inhibitory factor (oxMIF) which synergize with chemotherapeutic agents in animal models of cancer” (Cancer Research, 2014, 74 (19 Suppl) Abstract 2654).

Benonisson H. et al. report CD3/TYRP1/gp75 and CD3/HIV-1 gp120 bispecific antibodies (Molecular Cancer Therapeutics, 18(2), 2019, 312-322).

WO2019/234241 A1, published May 5, 2020 describes anti-oxMIF/anti-CD3 bispecific antibodies.

Klein C. et al. refer to the issue of chain association in the development of heterodimeric antibodies (MABS, 4(6), 2012, pp. 653-663).

An urgent need exists for solving the problem on how to develop an immune cell mediated therapy, which has enhanced specificity and effectiveness. Specifically, there is an unmet need for overcoming limitations of therapeutic antibodies such as anti-oxMIF antibodies in oncology.

### SUMMARY OF THE INVENTION

It is the objective of the invention to provide for a bispecific antibody format directed against oxMIF and CD3 with improved biological activity.

The object is solved by the subject matter as claimed.

According to the invention there is provided an anti-oxMIF/anti-CD3 antibody or antigen binding fragment thereof, comprising at least one binding site specifically recognizing oxMIF and one binding site specifically recognizing CD3, selected from the group consisting of
an IgG wherein a scFv is fused to only one of the two heavy chains,
an IgG wherein one Fab arm is replaced by a bispecific-T-cell-engager (BiTE) and one Fab arm is an IgG Fab arm and wherein said BiTE and IgG Fab arm are linked to the Fc-portion via the hinge region, and
an IgG wherein the Fab arms are replaced by scFvs with different specificities, comprising at least one binding site specifically recognizing oxMIF and one binding site specifically recognizing CD3.

The anti-oxMIF/anti-CD3 antibody of the invention has advantageous properties compared to the single antibody binding to oxMIF. Specifically, the bispecific formation of the inventive antibody brings tumor cells and T-cells in proximity to enable the T-cell to kill the tumor cells, thereby having the potential to significantly reduce tumor and metastasis burden.

According to a specific embodiment, the antibody induces T-cell-mediated cytotoxicity to a higher degree than the combination of anti-oxMIF and anti-CD3 antibodies. Such increase can be determined by any assay known in the art such as, but not limited to, a T cell Mediated Tumor Cell Lysis Assay. T-cell mediated cytotoxicity of the anti-oxMIF/anti-CD3 bispecific antibody may also be determined in vitro on cancer cells, specifically on solid tumor cells, specifically on colorectal, pancreatic, ovarian and lung cancer cells.

Specifically, the anti-oxMIF/anti-CD3 antibody described herein, having a scFv fused to only one of the two heavy chains offers the advantages of
a more balanced T cell activation due to a single anti-CD3 binding site,
a higher binding avidity to the target due to two anti-oxMIF binding sites and
a serum half life corresponding to a normal IgG due to its Fc-portion.

Specifically, the anti-oxMIF/anti-CD3 antibody described herein, wherein one Fab arm is replaced by a bispecific-T-cell-engager (BiTE) and one Fab arm is an IgG Fab arm and both are linked to the Fc-portion via the hinge region offers the advantages of
a more balanced T cell activation due to a single anti-CD3 binding site,
a higher binding avidity to target cells due to two anti-oxMIF binding sites and
a serum half life corresponding to a normal IgG due to its Fc-portion compared to BiTE.

According to the invention, the oxMIF binding site is specific for oxidized MIF and does not bind to reduced MIF.

According to the embodiment, the binding site of the herein described antibody or antigen binding fragment thereof comprises at least one binding site specifically recognizing oxMIF and one binding site specifically recognizing CD3, and the site specifically recognizing oxMIF comprises
(a) a variable CDR comprising sequences SEQ ID NOs 1 to 6, or a variable CDR region with at least 70% sequence identity to SEQ ID NOs 1 to 6, or
(b) a variable CDR comprising sequences SEQ ID NOs 7 to 12, or a variable CDR with at least 70% sequence identity to SEQ ID NOs 7 to 12, or
(c) a variable CDR comprising sequences SEQ ID NOs 13 to 18, or a variable CDR with at least 70% sequence identity to SEQ ID NOs 13 to 18, or
(d) a variable CDR comprising sequences SEQ ID Nos. 19 to 24, or a variable CDR with at least 70% sequence identity to SEQ ID Nos. 19 to 24, or
(e) a variable CDR comprising sequences SEQ ID NOs. 26, 27, 21, 28, 23, and 24, or a variable CDR with at least 70% sequence identity to SEQ ID NOs. 26, 27, 21, 28, 23, and 24, or
(f) a variable CDR comprising sequences SEQ ID NOs. 19, 20, 21, 138, 25, and 153, or a variable CDR with at least 70% sequence identity to SEQ ID NOs. 19, 20, 21, 138, 25, and 153.

According to a specific embodiment, the CDR sequences comprise 0, 1 or 2 point mutations.

In a further embodiment, the binding site of the anti oxMIF/anti-CD3 antibody described herein, which is specifically recognizing CD3, comprises a variable region comprising 0, 1, or 2 point mutations in each of the CDR sequences

SEQ ID NOs 77, 78, 149, 83, 84 and 151, or

SEQ ID NOs 77, 78, 79, 80, 81 and 82, or

SEQ ID NOs 77, 78, 79, 83, 84 and 85, or

SEQ ID NOs 77, 154, 79, 83, 84 and 85, or

SEQ ID NOs 86, 87, 88, 89, 90 and 91, or

SEQ ID NOs 92, 93, 94, 95, 96 and 97, or

SEQ ID NOs 167, 168, 169, 178, 179, and 180, or

SEQ ID NO 170, 171, 172, 181, 182 and 183.

According to a specific embodiment, the anti-oxMIF/anti-CD3 antibody described herein comprises 0 or 1 point mutation in the sequences SEQ ID NO 7, 8, 9, 10, 11, 12, 167, 168, 169, 178, 179 and 180.

According to a further embodiment, the anti-oxMIF/anti-CD3 antibody comprises the sequences SEQ ID NOs 7, 8, 9, 10, 11, 12, 77, 78, 149, 83, 84, and 151.

According to a specific embodiment, the IgG part of the anti-oxMIF/anti-CD3 antibody is recognizing oxMIF and the scFv fused to one of the heavy chains is recognizing CD3, further comprising a peptide linker joining the anti-CD3 variable light (VL) and variable heavy (VH) chains.

According to a further specific embodiment, the IgG Fab arm of the anti-oxMIF/anti-CD3 antibody is recognizing oxMIF and the bispecific T-cell engager (BiTE), replacing the second Fab-arm, is recognizing oxMIF and CD3. Said antibody is further comprising peptide linkers joining the VL and VH chains, i.e. interlink the VL with the VH chains, of the bispecific T-cell engager portion.

According to a further specific embodiment, the Fab arms of the anti-oxMIF/anti-CD3 antibody are replaced by scFvs, one scFv is targeting oxMIF and the other scFv is targeting CD3, said antibody further comprising peptide linkers joining the VL and VH chains.

Further provided herein is the anti-oxMIF/anti-CD3 antibody described herein, wherein the binding site specifically recognizing oxMIF comprises a heavy chain variable region having at least 80%, preferably at least 90%, more preferably at least 95%, more preferably at least 99% sequence identity to the amino acid sequence of SEQ ID NO 158, and a light chain variable region having at least 80%, preferably at least 90%, more preferably at least 95% sequence, more preferably at least 99% identity to the amino acid sequence of SEQ ID NO 134.

In a further embodiment, there is provided an anti-oxMIF/anti-CD3 antibody described herein, wherein the binding site specifically recognizing CD3 comprises a heavy chain variable region having at least 80%, preferably at least 90%, more preferably at least 95% sequence identity to the amino acid sequence of SEQ ID NO 135 and a light chain variable region having at least 80%, preferably at least 90%, more preferably at least 95% sequence identity to the amino acid sequence of SEQ ID NO 136.

According to yet a further embodiment, the anti-oxMIF/anti-CD3 antibody described herein comprises the amino acid sequence of SEQ ID NO 159, 137, 140, 160, 161, 162, 163, 194, 195, 196 or 197, or an amino acid sequence having at least 85%, 90%, specifically at least 95%, specifically at least 99% sequence identity with any one of SEQ ID NO 159, 137, 140, 160, 161, 162, 163, 194, 195, 196, or 197.

According to a specific embodiment, the invention specifically contemplates the use of any antibody comprising an oxMIF binding site derived from the sequences CDR1-H, CDR2-H, CDR3-H of the heavy chain variable region and/or the sequences CDR1-L, CDR2-L, CDR3-L of the light chain variable region, including constructs comprising single variable domains comprising either the combination of the CDR1-H, CDR2-H, CDR3-H sequences, or the combination of the CDR1-L, CDR2-L, CDR3-L sequences, or pairs of such variable domains, e.g. VH, VHH or VHNL domain pairs.

According to a specific embodiment, the invention specifically contemplates the use of any antibody comprising a CD3 binding site derived from the sequences CDR1-H, CDR2-H, CDR3-H of the heavy chain variable region and/or the sequences CDR1-L, CDR2-L, CDR3-L of the light chain variable region, including constructs comprising single variable domains comprising either the combination of the CDR1-H, CDR2-H, CDR3-H sequences, or the combination of the CDR1-L, CDR2-L, CDR3-L sequences, or pairs of such variable domains, e.g. VH, VHH or VHNL domain pairs.

A further specific embodiment refers to the anti-oxMIF/anti-CD3 antibody wherein the corresponding variable heavy chain regions (VH) and the corresponding variable light chain regions (VL) regions are arranged, from N-terminus to C-terminus, specifically in the order VL(oxMIF)-VH(oxMIF)-VH(CD3)-VL(CD3), VL(CD3)-VH(CD3)-VH(oxMIF)-VL(oxMIF), VH(CD3)-VL(CD3)-VL(oxMIF)-VH(oxMIF), VH(oxMIF)-VL(oxMIF)-VL(CD3)-VH(CD3), VL(oxMIF)-VH(oxMIF), VH(oxMIF)-VL(oxMIF), VH(CD3)-VL(CD3), or VL(CD3)-VH(CD3).

According to a further embodiment, the antibody comprises at least one antibody domain which is of human origin, or a chimeric, or humanized antibody domain of mammalian origin other than human, preferably of humanized, murine or camelid origin.

According to a further embodiment, the antibody as described herein comprises monovalent or bivalent binding portions specifically binding oxMIF, and a monovalent, binding portion specifically binding CD3.

According to a further embodiment of the invention, the Fc domain, specifically the CH3 domains, of the antibody described herein comprise knob-into-hole mutations known in the art (Ridgway J. B. B. et al., Protein Engineering, 1996, 617-621) or are produced by SEED technology (SEEDbodies, Davis J. H., et al., Protein Eng. Des. Sel., 2010, 23(4), 195-202).

According to a further embodiment, herein provided is also a pharmaceutical composition comprising the anti-oxMIF/anti-CD3 antibody and a pharmaceutically acceptable carrier or excipient.

Specifically, the antibody or the pharmaceutical composition as described herein is provided for use in the treatment of a hyperproliferative disorder, specifically cancer involving any tissue or organ, specifically in the treatment of head, neck, breast, liver, skin, gastric, bladder, renal, esophageal, gynecological, bronchial, nasopharynx, thyroid, prostate, colorectal, ovarian, pancreas, lung cancers, and fibrosarcoma.

Specifically, the antibody as described herein can be used as a medicament.

Specifically, a method for the treatment of a hypoproliferative disease, specifically cancer, is provided, comprising administering a therapeutically effective amount of a pharmaceutical composition as described herein to a subject in need thereof.

Further provided herein are isolated nucleic acid molecules encoding an anti-oxMIF/anti-CD3 antibody format of the invention.

In a further embodiment, there is provided an expression vector comprising nucleic acid molecule(s) as described herein.

A further embodiment refers to a host cell comprising said vector.

Further provided herein is a method of producing the anti-oxMIF/anti-CD3 antibody of the invention, comprising expressing a nucleic acid encoding the antibody in a host cell.

According to a specific embodiment, there is provided an in vitro method of detecting cellular expression of oxMIF, the method comprising: contacting a biological sample comprising a human cell to be tested with an anti-oxMIF/anti-CD3 antibody of the invention; and detecting binding of said antibody; wherein the binding of said antibody indicates the presence of oxMIF on a cell surface, to thereby detect whether the cell expresses oxMIF.

Specifically, the biological sample comprises intact human cells, biopsies, resections, tissue samples, or a membrane fraction of the cells to be tested.

More specifically, the anti-oxMIF/anti-CD3 antibody is labeled with a detectable label selected from the group consisting of a radioisotope, a fluorescent label, a chemiluminescent label, an enzyme label, and a bioluminescent label.

According to another aspect, the antibody conjugated to a detectable label can be used in diagnosing a hypoproliferative disease such as cancer, wherein the cells of a subject are expressing oxMIF.

### FIGURES

FIG. 1: Schematic picture of the anti-oxMIF/anti-CD3 bispecific antibody of oxMIF and CD3 that brings T cell in close proximity to tumor cell.

FIG. 2: Schematic picture of the antibody formats used in the examples. The figure shows an IgG wherein a scFv is fused to only one of the two heavy chains (IgG-scFv exemplified by C0086), an IgG wherein one Fab arm is replaced by a bispecific T-cell engager (Fab-BiTE-Fc, exemplified by C0061), and an IgG where both Fab-arms are replaced by scFvs with different binding specificities ((scFv)2-Fc or scFv(oxMIF)-scFv(CD3)-Fc; exemplified by C0062).

FIG. 3: Simultaneous binding of anti-oxMIF/CD3 bispecific antibodies C0061 and C0062 to oxMIF and CD3. The anti-oxMIF monospecific antibody C0008 is used as negative control.

FIG. 4: Binding of anti-oxMIF/CD3 bispecific antibodies C0061 and C0062 to immobilized oxMIF in an ELISA. The anti-oxMIF/CD3 monospecific antibody C0008 is used as positive control.

FIG. 5: Activation of T cells by anti-oxMIF/CD3 bispecific antibodies C0061 and C0062. The anti-oxMIF monospecific antibody C0008 is used as negative control.

FIG. 6: PBMC mediated tumor cell killing of HCT116 colon cancer cells using anti-oxMIF/CD3 bispecific antibodies C0061 and C0062. The anti-oxMIF monospecific antibody C0008 is used here as negative control.

FIG. 7: Simultaneous binding of anti-oxMIF/CD3 bispecific antibodies C0086 and C0107 to oxMIF and CD3. The anti-oxMIF monospecific antibody C0008 was used as negative control.

FIG. 8: Binding of anti-oxMIF/CD3 bispecific antibodies C0086 and C0107 to immobilized oxMIF in an ELISA. The anti-oxMIF monospecific antibody C0008 was used as positive control.

FIG. 9: Differential binding of the anti-oxMIF/CD3 bispecific antibodies (A) C0061, C0062, C0086 and (B) C0107 to oxMIF vs. redMIF. Imalumab (C0008) was used as reference antibody and a non-specific isoype IgG as negative control.

FIG. 10: Specific binding of anti-oxMIF/CD3 bispecific antibodies to native CD3 expressed on CD3-positive Jurkat T-cells, whereas only background staining was determined on CD3 negative Jurkat T-cells. The monospecific anti oxMIF antibody C0008 was used as negative control.

FIG. 11: IL-2 secretion of activated human T cells induced by anti-oxMIF/CD3 bispecific antibody C0061 (A) or monospecific anti-oxMIF antibody C0008 (B, negative control), either in the presence or in the absence of human HCT116 cancer target cells.

FIG. 12: PBMC mediated tumor cell killing of oxMIF displaying colon cancer cells HCT116 (A) and oxMIF displaying human ovarian cancer cells A2780 (B) using anti-oxMIF/CD3 bispecific antibodies. The anti-oxMIF monospecific antibody C0008 was used as negative control.

FIG. 13: Pharmacokinetics (PK) of C0061 in the circulation of NSG mice after intravenous injection.

FIG. 14: Tumor penetration and accumulation of C0061 by infra-red in vivo imaging of mice carrying subcutaneous CALU-6 tumors. Pictures were taken 1 h, 6 h, 24 h, 48 h, 72 h, 96 h and 168 h post injection of the IRDye 800CW labelled antibody. A: Mice which received IRDye 800CW-labeled C0061 (5 mg/kg); B: non-treated control mice; Scalebar is the same for A and B.

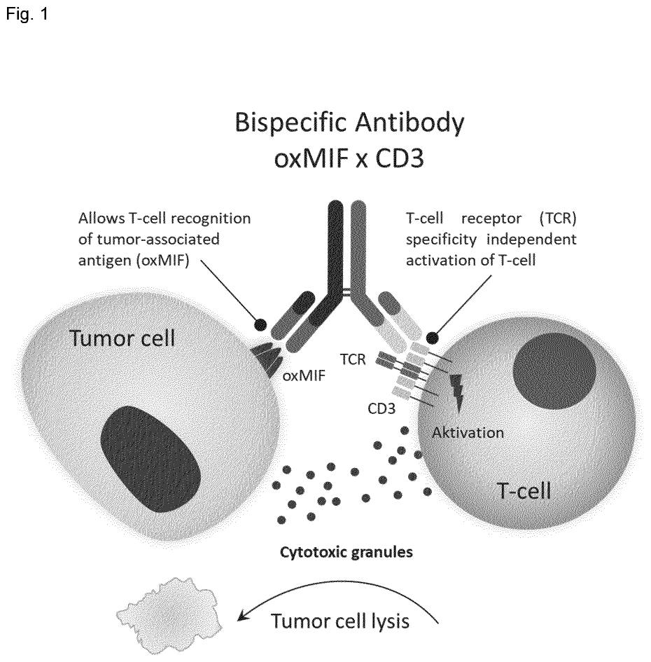

### DETAILED DESCRIPTION OF THE INVENTION

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

The antibody of the invention comprises at least one binding site specifically recognizing oxMIF and one binding site specifically recognizing CD3.

The oxMIF binding site is specific for the oxidized form of MIF, i.e. specifically for human oxMIF but does not show substantial cross-reactivity to reduced MIF. oxMIF is the disease-related structural isoform of MIF which can be specifically and predominantly detected in the circulation of subjects with inflammatory diseases and in tumor tissue of cancer patients.

The antibody of the invention further comprises one binding site specifically recognizing an epitope of CD3, specifically an epitope of human CD3, including the CD3γ (gamma) chain, CD3δ (delta) chain, and two CD3ε (epsilon) chains which are present on the cell surface. Clustering of CD3 on T cells, such as by immobilized anti-CD3 antibodies leads to T cell activation similar to the engagement of the T cell receptor but independent of its clone-typical specificity. In certain embodiments, the CD3 binding domain of the antibody described herein exhibits not only CD3 binding affinities with human CD3, but shows also excellent cross reactivity with the respective cynomolgus monkey CD3 proteins. In some instances, the CD3 binding domain of the antibody is cross-reactive with CD3 from cynomolgus monkey. Antibodies or fragments thereof that bind to CD3 with lower affinity can efficiently trigger T cell activation and cytotoxicity. This may be of increased therapeutic value because of their preferential localization to tumor cells. In one embodiment, the anti-CD3 binding site comprises one or more (e.g., all three) light chain complementary determining regions of an anti-CD3 binding domain described herein, and/or one or more (e.g., all three) heavy chain complementary determining regions of an anti-CD3 binding domain described herein, e.g., an anti-CD3 binding domain comprising one or more, e.g., all three, LC CDRs and one or more, e.g., all three, HC CDRs.

The term “antibody” herein is used in the broadest sense and encompasses polypeptides or proteins that consist of or comprise antibody domains, which are understood as constant and/or variable domains of the heavy and/or light chains of immunoglobulins, with or without a linker sequence. The term encompasses various antibody structures, including but not limited to monoclonal antibodies, polyclonal antibodies, multispecific antibodies such as bispecific antibodies, and antibody fragments as long as they exhibit the desired antigen-binding activity, i.e. binding to oxMIF and CD3 epitopes.

Antibody domains may be of native structure or modified by mutagenesis or derivatization, e.g. to modify the antigen binding properties or any other property, such as stability or functional properties, such as binding to the Fc receptors, such as FcRn and/or Fc-gamma receptor. Polypeptide sequences are considered to be antibody domains, if comprising a beta-barrel structure consisting of at least two beta-strands of an antibody domain structure connected by a loop sequence.

It is understood that the term “antibody” includes antigen binding derivatives and fragments thereof. A derivative is any combination of one or more antibodies or antibody domains of the invention and/or a fusion protein in which any domain of the antibody of the invention may be fused at any position of one or more other proteins, such as other antibodies or antibody formats, e.g. a binding structure comprising CDR loops, a receptor polypeptide, but also ligands, scaffold proteins, enzymes, labels, toxins and the like.

The term “antibody and antigen binding fragment thereof” shall particularly refer to polypeptides or proteins that exhibit bispecific binding properties, i.e. to the target antigens oxMIF and CD3.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, Fab-scFv fusion, Fab-(scFv)₂-fusion, Fab-scFv-Fc, F(ab′)₂, ScFvFc, diabodies, cross-Fab fragments; linear antibodies; single-chain antibody molecules (e.g. scFv); and multispecific antibodies formed from antibody fragments. In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies. Antibody fragments as referred herein also encompass Fc domains comprising one or more structural loop regions containing antigen binding regions such as Fcab™ or full length antibody formats with IgG structures in which the Fc region has been replaced by an Fcab containing second distinct antigen binding site.

As used herein, “Fab fragment or Fab” refers to an antibody fragment comprising a light chain comprising a VL domain and a constant domain of a light chain (CL), and a VH domain and a first constant domain (CH1) of a heavy chain.

As used herein, “Fab arm” refers to a Fab fragment linked to an Fc-portion or Fc-domains by a hinge region.

The term “N-terminus” denotes the last amino acid of the N-terminus.

The term “C-terminus” denotes the last amino acid of the C-terminus.

A “BiTE” of “bi-specific T-cell engager” refers to an artificial monoclonal antibody which is a fusion protein consisting of two single-chain variable fragments (scFvs) of different antibodies, or amino acid sequences from four different genes, on a single peptide chain of about 50 kilodaltons. One of the scFvs binds to a T cell via the CD3 receptor, and the other to a tumor cell via oxMIF.

In a specific embodiment, the term Fab-BiTE-Fc refers to an anti-oxMIF/anti-CD3 antibody which is an IgG having one Fab arm replaced by a bispecific-T-cell-engager (BiTE), while the second IgG arm is preserved. In a specific embodiment the Fab-BiTE-Fc specifically comprises the sequence or a sequence with at least 70%, specifically 75%, 80%, 85%, 90%, 95/or 99% sequence identity with SEQ ID NO 137:

DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKGGGGSGGGGSGGGGSEVQLLESGGGLVQPGGSLRLSCAASGFT
FSIYSMNWVRQAPGKGLEWVSSIGSSGGTTYYADSVKGRFTISRDNSKNT
LYLQMNSLRAEDTAVYYCAGSQWLYGMDVWGQGTTVTVSSGGGGSQVQLV
QSGAEVKKPGASVKVSCKASGYTFTRYTMHWVRQAPGQGLEWMGYINPSR
GYTNYNQKFKDRVTLTTDKSSSTAYMELSSLRSEDTAVYYCARYYDDHYS
LDYWGQGTLVTVSSGGSGGSGGSGGSGGSDIQMTQSPSSLSASVGDRVTI
TCSASSSVSYMNWYQQKPGKAPKRLIYDTSKLASGVPSRFSGSGSGTDFT
LTISSLQPEDFATYYCQQWSSNPFTFGQGTKLEIKGGGGSDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSPGK (SEQ ID NO 137, polypeptide 2 of
C0061).

Specifically, the FaB-BiTE comprises SEQ ID NOs 137, 159 and 140, specifically comprises the sequence or a sequence with at least 70%, specifically 75%, 80%, 85%, 90%, 95/or 99% sequence identity with SEQ ID NOs 137, 159 and 140.

This leads to a longer half life in comparison to known BiTEs.

The term “IgG-scFv” refers to a kind of bispecific antibodies which is engineered for bispecificity by fusing one scFv to a monospecific Immunoglobulin G (IgG). The specificity of the IgG can be for oxMIF and the specificity of the scFv can be for CD3 or vice versa. Furthermore, either the amino terminus or the C terminus of one of the light or heavy chains can be appended with an scFv, which leads to the production of diverse types of IgG-scFv bispecific antibodies (BsAbs): (i) IgG(H)-scFv, an scFv linked to the C terminus of one of the full-length IgG HC; (ii) scFv-(H)IgG, which is same like IgG(H)-scFv, except that the scFv is linked to the HC N terminus. (iii) IgG(L)-scFv or (iv) scFv-(L)IgG: the scFv connected to the C or N terminus of the IgG light chain, which forms the IgG(L)-scFv or scFv-(L)IgG, respectively. Specifically, the IgG-scFv is in the range of 165 kDa to 185 kDa, specifically it is about 175 kDa.

In a specific embodiment, the IgG-scFv (anti-oxMIF IgG x anti-CD3scFv fusion protein) of the invention comprises the sequence or a sequence with at least 70%, specifically 75%, 80%, 85%, 90%, 95/or 99% sequence identity with SEQ ID NO 139 and/or SEQ ID NO 140:

EVQLLESGGGLVQPGGSLRLSCAASGFTFSIYSMNWVRQAPGKGLEWVSS
IGSSGGTTYYADSVKGRFTISRDNSKNTLYLQMNSLRAEDTAVYYCAGSQ
WLYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVC
TLPPSRDELTKNQVSLSCAVKGFYPSDIAVEWESNGQPENNYKTTPPVLD
SDGSFFLVSKLTVDKSRWQQGNVFSCSVMHEALHNHYTQKSLSLSPGKGG
GGSGGGGSGGGGSQVQLVQSGAEVKKPGASVKVSCKASGYTFTRYTMHWV
RQAPGQGLEWMGYINPSRGYTNYNQKFKDRVTLTTDKSSSTAYMELSSLR
SEDTAVYYCARYYDDHYSLDYWGQGTLVTVSSGGSGGSGGSGGSGGSDIQ
MTQSPSSLSASVGDRVTITCSASSSVSYMNWYQQKPGKAPKRLIYDTSKL
ASGVPSRFSGSGSGTDFTLTISSLQPEDFATYYCQQWSSNPFTFGQGTKL
EIK (SEQ ID NO 139, anti-oxMIF heavy chain -
anti-CD3 scFv fusion, polypeptide 1 of C0086).
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO 140, anti-oxMIF light
chain, polypeptide 3 of C0086 and C0061).

Specifically, the IgG-scFv (anti-oxMIF IgG x anti-CD3scFv fusion protein) comprises SEQ ID NOs 139, 140 and 163, specifically comprises the sequence or a sequence with at least 70%, specifically 75%, 80%, 85%, 90%, 95/or 99% sequence identity with SEQ ID NOs 139, 140 and 163.

In another specific embodiment the term (scFv)₂-Fc refers to an IgG having one Fab arm being replaced by an anti-oxMIF scFv and the other Fab arm being replaced by an anti-CD3 scFv. In a specific embodiment, the (scFv)₂-Fc comprises the sequence or a sequence with at least 70%, specifically 75%, 80%, 85%, 90%, 95/or 99% sequence identity to SEQ ID NO 156 or SEQ ID NO 157:

DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKGGGGSGGGGSGGGGSEVQLLESGGGLVQPGGSLRLSCAASGFT
FSIYSMNWVRQAPGKGLEWVSSIGSSGGTTYYADSVKGRFTISRDNSKNT
LYLQMNSLRAEDTAVYYCAGSQWLYGMDVWGQGTTVTVSSGGGGSDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPCRDELTKNQVSLWCLVKGFYPS
DIAVEWESNGQPENNYKTTPPVLDSDGSFFLYSKLTVDKSRWQQGNVFSC
SVMHEALHNHYTQKSLSLSPGK (SEQ ID NO 156, Polypeptide
1 of C0062 without Strep tag).
QVQLVQSGAEVKKPGASVKVSCKASGYTFTRYTMHWVRQAPGQGLEWMGY
INPSRGYTNYNQKFKDRVTLTTDKSSSTAYMELSSLRSEDTAVYYCARYY
DDHYSLDYWGQGTLVTVSSGGSGGSGGSGGSGGSDIQMTQSPSSLSASVG
DRVTITCSASSSVSYMNWYQQKPGKAPKRLIYDTSKLASGVPSRFSGSGS
GTDFTLTISSLQPEDFATYYCQQWSSNPFTFGQGTKLEIKGGGGSDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVCTLPPSRDELTKNQVSLSCAVKGFYPS
DIAVEWESNGQPENNYKTTPPVLDSDGSFFLVSKLTVDKSRWQQGNVFSC
SVMHEALHNHYTQKSLSLSPGK (SEQ ID NO 157, Polypeptide
2 of C0062 without His tag).

According to a specific embodiment, the antibodies described herein may comprise one or more tags for purification and/or detection, such as but not limited to affinity tags, solubility enhancement tags and monitoring tags.

Specifically, the affinity tag is selected from the group consisting of poly-histidine tag, poly-arginine tag, peptide substrate for antibodies, chitin binding domain, RNAse S peptide, protein A, β-galactosidase, FLAG tag, Strep II tag, streptavidin-binding peptide (SBP) tag, calmodulin-binding peptide (CBP), glutathione S-transferase (GST), maltose-binding protein (MBP), S-tag, HA tag, and c-Myc tag, specifically the tag is a His tag comprising one or more H, more specifically it is a hexahistidine tag.

Affinity tags may be attached to any domain of the antibody described herein, specifically to Fc moieties, more specifically to the CH3 domains or to Fab domains, specifically to VL.

By “fused” or “connected” is meant that the components (e.g. a Fab molecule and an Fc domain subunit) are linked by peptide bonds, either directly or via one or more peptide linkers.

The term “linker” as used herein refers to a peptide linker and is preferably a peptide with an amino acid sequence specifically consisting of 1, 2, 3, 4, 5, 6, 7, 8, 9, 10, 11, 12, 13, 14, 15, 16 or more amino acid residues, specifically the linker consists of 5 amino residues or repeated units of 5 amino acids. The peptides designed for connecting the individual domains preferably do not interfere with the folding of the connected domains. Specifically, the linker sequence comprises glycine and/or serine residues, more specifically the linker is (GGS)ₙ, or (GGGS)ₙ (SEQ ID NO 166) wherein n is 1, 2, 3 or more.

Said peptide linkers may specifically connect VH and VL chains of the antibodies or antigen binding moieties described herein. Peptide linkers may also connect two VH sequences of different binding sites, such as CD3 VH and oxMIF VH.

Specifically, when the IgG is recognizing oxMIF and the scFv is recognizing CD3, the peptide linkers joining the anti-CD3 variable light (VL) and variable heavy (VH) comprise the sequence GGGGS (SEQ ID NO 164) or GGS or repeated sequences thereof, specifically (GGGGS)ₙ wherein n is 1, 2, 3 or more, specifically n is 3 or (GGS)ₙ, wherein n is 1, 2, 3, 4, 5, or more, specifically n is 5.

In an alternative embodiment, when the IgG Fab arm is recognizing oxMIF and the bispecific T-cell engager is recognizing oxMIF and CD3, peptide linkers joining the VL and VH domains of oxMIF and CD3 binding regions are of the sequence (GGGS)ₙ or (GGGGS)ₙ, or any combinations thereof, wherein n is 1, 2, 3, 4, 5 or more. Peptide linkers may also be present for connecting the oxMIF VH and CD3 VH, specifically comprising the sequences GGSGGS (SEQ ID NO 165), (GGS)ₙ or (GGGGS)ₙ, wherein n is 1, 2, 3, 5 or more.

In a further embodiment when both Fab arms are replaced by scFvs and one scFv is targeting oxMIF and the other scFV is targeting CD3, peptide linkers joining the anti-CD3 VL and VH chains specifically can have the sequence (GGS)ₙ, wherein n is 1, 2, 3, 4, 5, specifically n is 5. Peptide linkers can also connect the Fc arm with anti-CD3 VH, specifically comprising the sequence (GGGGS)ₙ, wherein n is 1,2, 3 or more, specifically n is 3.

The term “immunoglobulin” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. An immunoglobulin of the IgG class essentially consists of two Fab molecules (Fab arms) and an Fc domain, linked via the immunoglobulin hinge region. The heavy chain of an immunoglobulin may be assigned to one of five types, called a (IgA), δ (IgD), ε (IgE), γ (IgG), or μ (IgM), some of which may be further divided into subtypes, e.g. γ₁ (IgG₁), γ₂ (IgG2), γ₃ (IgG3), γ₄ (IgG4), α₁ (IgA₁) and α₂ (IgA₂). The light chain of an immunoglobulin may be assigned to one of two types, called kappa (κ) and lambda (λ).

The term “chimeric antibody” refers to an antibody in which a portion of the heavy and/or light chain is derived from a particular source or species, while the remainder of the heavy and/or light chain is derived from a different source or species, usually prepared by recombinant DNA techniques. Chimeric antibodies may comprise a rabbit or murine variable region and a human constant region. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art (Morrison, S. L., et al., Proc. Natl. Acad. Sci. 81 (1984) 6851-6855).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. As for chimeric and humanized antibodies, the term “human antibody” as used herein also comprises such antibodies which are modified in the constant region e.g. by “class switching” i.e. change or mutation of Fc parts (e.g. from IgG1 to IgG4 and/or IgG1/IgG4 mutation).

The term “recombinant human antibody”, as used herein, is intended to include all human antibodies that are prepared, expressed, created or isolated by recombinant means, such as antibodies isolated from a host cell such as a HEK cell, NS0 or CHO cell or from an animal (e.g. a mouse) that is transgenic for human immunoglobulin genes or antibodies expressed using a recombinant expression vector transfected into a host cell. The amino acid sequences of the VH and VL regions of the recombinant antibodies are sequences that, while derived from and related to human germ line VH and VL sequences, may not naturally exist within the human antibody germ line repertoire in vivo.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as described in Kabat et al., Sequences of Proteins of Immunological Interest, Fifth Edition, NIH Publication 91-3242, Bethesda Md. (1991), vols. 1-3.

A “humanized” antibody refers to a chimeric antibody comprising amino acid residues from non-human HVRs and amino acid residues from human framework regions (FRs) which has undergone humanization. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. Other forms of humanized antibodies encompassed by the present invention are those in which the constant region has been additionally modified or changed from that of the original antibody to generate the new properties, e.g. in regard to Cl q binding and/or Fc receptor (FcR) binding.

“Bispecific antibodies (Bsab)” according to the invention are antibodies which have two different binding specificities. Antibodies of the present invention are specific for oxMIF and CD3. The term bispecific antibody as used herein denotes an antibody or derivative or fragment thereof that has one or two binding sites for oxMIF and one binding site for CD3. Examples of bispecific antibody formats can be, but are not limited to bispecific IgGs (BsIgG), IgGs appended with an additional antigen-binding moiety, BsAb fragments, bispecific fusion proteins, BsAb conjugates, hybrid BsIgGs, variable domain only bispecific antibody molecules, CH1/CL fusion proteins, Fab fusion proteins, modified Fc and CH3 fusion proteins, appended IgGs-HC fusions, appended IgGs-LC fusions, appended IgGs-HC&LC fusions, Fc fusions, CH3 fusions, IgE/IgM CH2 fusions, F(ab′)2 fusions, CH1/CL, modified IgGs, non-immunoglobulin fusion proteins, Fc-modified IgGs, diabodies, etc. as described in Spiess C. et al., 2015, Mol. Immunol., 67, 95-106 and Brinkmann U. and Kontermann R. E., 2017, MABS, 9, 2, 182-212).

The Fc-portion can be modified to comprise knob-into-hole mutations to engineer CH3 for heterodimerization. Knobs are created by replacing small amino side chains at the interface between CH3 domains with larger ones, holes are constructed by replacing large side chains with smaller ones. Specifically, one Fc arm can comprise mutations S354C and T366W, the other Fc arm can comprise mutations Y349C, T366S, L368A, Y407V according to the EU numbering scheme. As an alternative, the strand-exchange engineered domain (SEED) technology can be used for modifying the Fc arms to generate the asymmetric and bispecific antibody-like molecules. The technology is based on exchanging structurally related sequences of the immunoglobulin within the conserved CH3 domains. Alternating sequences from human IgA and IgG in the SEED CH3 domains can generate two asymmetric but complementary domains, designated AG and GA. The SEED design allows efficient generation of AG/GA heterodimers, while disfavoring homodimerization of AG and GA SEED CH3 domains (Muda M. et al., 2011, Protein Eng. Des. Sel., 24(5), 447-54).

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitopes, which are immunologically relevant, may be recognized by such binding site.

The term “epitope” as used herein shall in particular refer to a molecular structure which may completely make up a specific binding partner or be part of a specific binding partner to a binding site of an antibody format of the present invention. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and specifically less than 10 amino acids, specifically between 4-10 amino acids. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding the polypeptide to form a tertiary structure and the amino acids are not necessarily adjacent to one another in the linear sequence. Such oxMIF epitope may be sequence EPCALCS (SEQ ID NO 145) located within the central region of oxMIF. However, the epitope may also be on the C-terminus of oxMIF.

The term “antigen binding domain” or “binding domain” or “binding-site” refers to the part of an antigen binding moiety that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “binding site” as used herein with respect to the antibody of the present invention refers to a molecular structure capable of binding interaction with an antigen. Typically, the binding site is located within the complementary determining region (CDR) of an antibody, herein also called “a CDR binding site”, which is a specific region with varying structures conferring binding function to various antigens. The varying structures can be derived from natural repertoires of antibodies, e.g. murine or human repertoires, or may be recombinantly or synthetically produced, e.g. by mutagenesis and specifically by randomization techniques. These include mutagenized CDR regions, loop regions of variable antibody domains, in particular CDR loops of antibodies, such as CDR1, CDR2 and CDR3 loops of any of VL and/or VH antibody domains. The antibody format as used according to the invention typically comprises one or more CDR binding sites, each specific to an antigen.

The term “recognizing”, “targeting” or “binding” can be used interchangeably herein.

The term “specific” or “bispecific” as used herein shall refer to a binding reaction which is determinative of the cognate ligand of interest in a heterogeneous population of molecules.

Herein, the binding reaction is at least with a CD3 antigen and an oxMIF antigen. Thus, under designated conditions, e.g. immunoassay conditions, the antibody that specifically binds to its particular targets does not bind in a significant amount to other molecules present in a sample, specifically it does not show detectable binding to reduced MIFA specific binding site is typically not cross-reactive with other targets. Still, the specific binding site may specifically bind to one or more epitopes, isoforms or variants of the target, or be cross-reactive to other related target antigens, e.g., homologs or analogs.

The specific binding means that binding is selective in terms of target identity, high, medium or low binding affinity or avidity, as selected. Selective binding is usually achieved if the binding constant or binding dynamics to a target antigen such as oxMIF and CD3 is at least 10 fold different, preferably the difference is at least 100 fold, and more preferred a least 1000 fold compared to binding constant or binding dynamics to an antigen which is not the target antigen.

The bispecific antibody of the present invention specifically comprises two or three sites with specific binding properties, wherein two different target antigens, CD3 and oxMIF, are recognized by the antibody. Thus, an exemplary bispecific antibody format may comprise two binding sites, wherein each of the binding sites is capable of specifically binding a different antigen, CD3 and oxMIF or three binding sites, wherein two binding sites bind to oxMIF and one binding site to CD3.

The term “valent” as used within the current application denotes the presence of a specified number of binding sites in an antibody molecule. As such, the terms “bivalent”, “tetravalent”, and “hexavalent” denote the presence of two binding sites, four binding sites, and six binding sites, respectively, in an antibody molecule.

The bispecific antibodies according to the invention are at least “bivalent” and may be “trivalent”.

The term “monovalent” as used herein with respect to a binding site of an antibody shall refer to a molecule comprising only one binding site directed against a target antigen.

Specifically, the antibody of the present invention is understood to be monovalent or bivalent for oxMIF and monovalent for CD3, thus either bivalent or trivalent in total.

According to a further embodiment, the antibody can comprise one or more additional binding sites specifically recognizing one or more antigens expressed on the effector T cells, specifically one or more of ADAM17, CD2, CD4, CD5, CD6, CD8, CD11a, CD11b, CD14, CD16, CD16b, CD25, CD28, CD30, CD32a, CD40, CD 40L, CD44, CD45, CD56, CD57, CD64, CD69, CD74, CD89, CD90, CD137, CD177, CEAECAM6, CEACAM8, HLA-Dra cahin, KIR, LSECtin or SLC44A2.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition (Kabat et al., 1991, Sequences of Proteins of Immunological Interest, 5th Ed. Public Health Service, National Institutes of Health, Bethesda, Md.) Hypervariable regions (HVRs) are also referred to as complementarity determining regions (CDRs), and these terms are used herein interchangeably in reference to portions of the variable region that form the antigen binding regions. The exact residue numbers which encompass a particular CDR will vary depending on the sequence and size of the CDR. Those skilled in the art can routinely determine which residues comprise a particular CDR given the variable region amino acid sequence of the antibody.

Kabat defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., 1983, U.S. Dept. of Health and Human Services, “Sequence of Proteins of Immunological Interest”. Unless otherwise specified, references to the numbering of specific amino acid residue positions in an antibody variable region are according to the Kabat numbering system. In a specific embodiment, the numbering of the constant region is according to EU numbering index.

CDRs also comprise “specificity determining residues,” or “SDRs,” which are residues that contact antigen. SDRs are contained within regions of the CDRs called abbreviated-CDRs, or a-CDRs. Unless otherwise indicated, HVR residues and other residues in the variable domain (e.g., FR residues) are numbered herein according to Kabat et al., supra.

According to a specific embodiment, the anti-CD3 binding site comprises complementary determining regions (CDRs) selected from the group consisting of muromonab-CD3 (OKT3), otelixizumab (TRX4), teplizumab (MGA031), visilizumab (Nuvion), foralumab, solitomab, blinatumomab, pasotuxizumab, cibisatamab SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31 and any humanized derivatives thereof, if applicable.

The antibody of the invention specifically comprises one or more of the sequences as described below:

TABLE 1
anti-oxMIF heavy chain sequences

| | HV- | | | | | |
|---|---|---|---|---|---|---|
| | CDR1 | | HV-CDR2 | | | |
| | (CDR1- | | (CDR2- | | HV-CDR3 | HV- |
| HV-FR1 | H1) | HV-FR2 | H1) | HV-FR3 | (CD3-H1) | FR4 |
| EVQLLESGGG | IYTM | WVRQA | YISPSGG | RFTISRDNSKNTL | RQYVLRY | WGQ |
| LVQPGGSLRL | D | PGKGLE | NTSYADS | YLQMNSLRAEDT | FDWSAD | GTMV |
| SCAASGFTFS | SEQ | WVS | VKG SEQ | AVYYCAS | AFDI | TVSS |
| SEQ ID NO 29 | ID NO | SEQ ID | ID NO 2 | SEQ ID NO 31 | SEQ ID NO | SEQ ID |
| | 1 | NO 30 | | | 3 | NO 32 |
| EVQLLESGGG | IYSM | WVRQA | SIGSSGG | RFTISRDNSKNTL | SQWLYG | WGQG |
| LVQPGGSLRL | N | PGKGLE | TTYYADS | YLQMNSLRAEDT | MDV | TTVTV |
| SCAASGFTFS | SEQ | WVS | VKG | AVYYCAG | SEQ ID NO | SS |
| SEQ ID NO 37 | ID NO | SEQ ID | SEQ ID | SEQ ID NO 39 | 9 | SEQ ID |
| | 7 | NO 38 | NO 8 | | | NO 40 |
| EVQLLESGGG | KYY | WVRQA | WIGPSG | RFTISRDNSKNTL | GTPDYG | WGQG |
| LVQPGGSLRL | MI | PGKGL | GFTFYA | YLQMNSLRAEDT | GNSLDH | TLVTV |
| SCAASGFTFS | SEQ | EVWS | DSVKG | AVYYCAR | SEQ ID NO | SS |
| SEQ ID NO 45 | ID NO | SEQ ID | SEQ ID | SEQ ID NO 47 | 15 | SEQ ID |
| | 13 | NO 46 | NO 14 | | | NO 48 |
| EVQLLESGGG | IYAM | WVRQA | GIVPSGG | RFTISRDNSKNTL | VNVIAVA | WGQ |
| LVQPGGSLRL | D | PGKGL | FTKYADS | YLQMNSLRAEDT | GTGYYYY | GTTV |
| SCAASGFTFS | SEQ | EWVS | VKG | AVYYCAR | GMDV | TVSS |
| SEQ ID NO 53 | ID NO | SEQ ID | SEQ ID | SEQ ID NO 55 | SEQ ID NO | SEQ ID |
| | 19 | NO 54 | NO 20 | | 21 | NO 56 |
| EVQLLESGGG | IYAM | WVRQA | GIVPSGG | RFTISRDNSKNTL | VNVIAVA | WGQ |
| LVQPGGSLRL | D | PGKGL | FTKYADS | YLQMNSLRAEDT | GTGYYYY | GTTV |
| SCAASGFTFS | SEQ | EVWS | VKG | AVYYCAR | GMDV | TVSS |
| SEQ ID NO 61 | ID NO | SEQ ID | SEQ ID | SEQ ID NO 63 | SEQ ID NO | SEQ ID |
| | 19 | NO 62 | NO 20 | | 21 | NO 64 |
| EVQLLESGGG | WYA | WVRQA | GIYPSGG | RFTISRDNSKNTL | VNVIAVAG | WGQG |
| LVQPGGSLRL | MD | PGKGL | RTKYAD | YLQMNSLRAEDT | TGYYYYG | TTVTV |
| SCAASGFTFS | SEQ | EVWS | SVKG | AVYYCAR | MDV | SS |
| SEQ ID NO 69 | ID NO | SEQ ID | SEQ ID | SEQ ID NO 71 | SEQ ID NO | SEQ ID |
| | 26 | NO 70 | NO 27 | | 21 | NO 72 |

TABLE 2
anti-oxMIF light chain sequences

| | LV- | | LV- | | LV- | |
|---|---|---|---|---|---|---|
| | CDR1 | | CDR2 | | CDR3 | |
| | (CDR1- | | (CDR2- | | (CDR3- | |
| LV-FR1 | LD | LV-FR2 | L1) | LV-FR3 | L1) | LV-FR4 |
| DIQMTQSPSS | RASQSI | WYQQKP | AASSLQ | GVPSRFSGSG | QQSYST | FGQGTK |
| LSASVGDRVT | SSYLN | GKAPKLL | S | SGTDFTLTISSL | PWT | VEIK SEQ |
| ITC SEQ ID | SEQ ID | IY SEQ ID | SEQ ID | QPEDFATYYC | SEQ ID | ID NO 36 |
| NO 33 | NO 4 | NO 34 | NO 5 | SEQ ID NO 35 | NO 6 | |
| DIQMTQSPSS | RSSQRI | WYQQKP | VASHSQ | GVPSRFRGSG | QQSFW | FGGGTK |
| LSASVGDRVT | MTYLN | GKAPKLL | S | SETDFTLTISGL | TPLT | VEIK SEQ |
| ITC SEQ ID | SEQ ID | IF SEQ ID | SEQ ID | QPEDSATYYC | SEQ ID | ID NO 44 |
| NO 41 | NO 10 | NO 42 | NO 11 | SEQ ID NO 43 | NO 12 | |
| DIQMTQSPSS | RASQSI | WYQHKP | ATSRLQ | GVPSRFSGGG | QQTYST | FGGGTK |
| LPASVGDRVT | GTYLS | GNAPKLL | S | SGTRFTLAISSL | PLT | VDIK SEQ |
| ITC SEQ ID | SEQ ID | IY SEQ ID | SEQ ID | QPDDFATYFC | SEQ ID | ID NO 52 |
| NO 49 | NO 16 | NO 50 | NO 17 | SEQ ID NO 51 | NO 18 | |
| DIQMTQSPGT | RASQG | WYQQKP | GTSSRA | GIPDRFSGSAS | QQYGR | FGGGTK |
| LSLSPGERAT | VSSSSL | GQAPRLL | T | GTDFTLTISRL | SLT | VEIK SEQ |
| LSC SEQ ID | A SEQ | IY SEQ ID | SEQ ID | QPEDFAVYYC | SEQ ID | ID NO 60 |
| NO 57 | ID NO 22 | NO 58 | NO 23 | SEQ ID NO 59 | NO 24 | |
| DIQMTQSPVT | RASQSV | WYQQKP | GASNR | GIPDRFSGSGS | QQYGN | FGGGTK |
| LSLSPGERAT | RSSYLA | GQTPRLL | AT | GTDFTLTISRLE | SLT | VEIK SEQ |
| LSC SEQ ID | SEQ ID | IY SEQ ID | SEQ ID | PEDFAVYYC | SEQ ID | ID NO 68 |
| NO 65 | NO 138 | NO 66 | NO 25 | SEQ ID NO 67 | NO 153 | |
| DIQMTQSPGT | RASQG | WYQQKP | GTSSRA | GIPDRFSGSAS | QQYGR | FGGGTK |
| LSLSPGERAT | VSSSSL | GQAPRLL | T | GTDFTLTISRL | SLT | VEIK SEQ |
| LSC SEQ ID | A SEQ | IY SEQ ID | SEQ ID | QPEDFAVYYC | SEQ ID | ID NO 76 |
| NO 73 | ID NO 28 | NO 74 | NO 23 | SEQ ID NO 75 | NO 24 | |

TABLE 3
anti-CD3 heavy chain sequences

| | HV- | | | | HV- | |
|---|---|---|---|---|---|---|
| | CDR1 | | | | CDR3 | |
| | (CDR1- | | HV-CDR2 | | (CDR3- | |
| HV-FR1 | H2) | HV-FR2 | (CDR2-H2) | HV-FR3 | H2) | HV-FR4 |
| QVQLVQSGAE | RYTM | WVRQAP | YINPSRG | RVTLTTDKSSST | YYDDH | WGQGT |
| VKKPGASVKV | H | GQGLE | YTNYNQK | AYMELSSLRSED | YSLDY | LVTVSS |
| SCKASGYTFT | SEQ ID | WMG | FKD | TAVYYCAR | SEQ ID | SEQ ID |
| SEQ ID NO 146 | NO 77 | SEQ ID | SEQ ID NO | SEQ ID NO 148 | NO 149 | NO 155 |
| | | NO 147 | 78 | | | |
| DIKLQQSGAEL | RYTM | WVKQRP | YINPSRG | KATLTTDKSSST | YYDDH | WGQGT |
| ARPGASVKMS | H SEQ | GQGLE | YTNYNQK | AYMQLSSLTSED | YCLDY | TLTVSS |
| CKTSGYTFT | ID NO | WIG | FKD | SAVYYCAR | SEQ ID | SEQ ID |
| SEQ ID NO 98 | 77 | SEQ ID | SEQ ID NO | SEQ ID NO 100 | NO 79 | NO 101 |
| | | NO 99 | 78 | | | |
| QVQLQQSGAE | RYTM | WVKQRP | YINPSRG | KATLTTDKSSST | YYDDH | WGQGT |
| LARPGASVKM | H SEQ | GQGLE | YTNYNQK | AYMQLSSLTSED | YCLDY | TLTVSS |
| SCKASGYTFT | ID NO | WIG | FKD | SAVYYCAR | SEQ ID | SEQ ID |
| SEQ ID NO 106 | 77 | SEQ ID | SEQ ID NO | SEQ ID NO 100 | NO 79 | NO 101 |
| | | NO 99 | 78 | | | |
| QVQLVQSGGG | RYTM | WVRQAP | YINPSRG | RFTISRDNSKNT | YYDDH | WGQGT |
| WQPGRSLRL | H SEQ | GKGLEW | YTNYNQK | AFLQMDSLRPED | YCLDY | PVTVSS |
| SCKASGYTFT | ID NO | IG | VKD | TGVYFCAR | SEQ ID | SEQ ID |
| SEQ ID NO 110 | 77 | SEQ ID | SEQ ID NO | SEQ ID NO 112 | NO 79 | NO 113 |
| | | NO 111 | 154 | | | |
| QVQLVESGGG | GYGM | WVRQAP | VIWYDGS | RFTISRDNSKNT | QMGY | WGRGT |
| WQPGRSLRL | H SEQ | GKGLEW | KKYYVDS | LYLQMNSLRAED | WHFDL | LVTVSS |
| SCAASGFKFS | ID NO | VA | VKG | TAVYYCAR | SEQ ID | SEQ ID |
| SEQ ID NO 118 | 86 | SEQ ID | SEQ ID NO | SEQ ID NO 120 | NO 88 | NO 121 |
| | | NO 119 | 87 | | | |
| EVQLLESGGG | SFPMA | WVRQAP | TISTSGG | RFTISRDNSKNT | FRQYS | WGQGT |
| LVQPGGSLRL | SEQ ID | GKGLEW | RTYYRDS | LYLQMNSLRAED | GGFDY | LVTVSS |
| SCAASGFTFS | NO 92 | VS | VKG | TAVYYCAK | SEQ ID | SEQ ID |
| SEQ ID NO 126 | | SEQ ID | SEQ ID NO | SEQ ID NO 128 | NO 94 | NO 129 |
| | | NO 127 | 93 | | | |
| EVQLVESGGG | GFTFN | WVRQAP | RIRSKYN | RFTISRDDSKNT | VRHGN | WGQGT |
| LVQPGGSLKL | KYAM | GKGLEW | NYATYYA | AYLQMNNLKTED | FGNSY | LVTVSS |
| SCAAS | N | VA | DSVKDS | TAVYYC | ISYWA | SEQ ID |
| SEQ ID NO 173 | SEQ ID | SEQ ID | SEQ ID NO | SEQ ID NO 174 | Y | NO 129 |
| | NO 167 | NO 119 | 168 | | SEQ ID | |
| | | | | | NO 169 | |
| EVQLLESGGG | GFTFS | WVRQAP | RIRSKYN | RFTISRDDSKNT | VRHGN | WGQGT |
| LVQPGGSLRL | TYAM | GKGLEW | NYATYYA | LYLQMNSLRAED | FGNSY | LVTVSS |
| SCAAS | N | VS | DSVKG | TAVYYC | VSWFA | SEQ ID |
| SEQ ID NO 175 | SEQ ID | SEQ ID | SEQ ID NO | SEQ ID NO 177 | Y | NO 129 |
| | NO 170 | NO 176 | 171 | | SEQ ID | |
| | | | | | NO 172 | |

TABLE 4
anti-CD3 light chain sequences

| | LV- | | | | | |
|---|---|---|---|---|---|---|
| | CDR1 | | | | | |
| | (CDR1- | | LV-CDR2 | | LV-CDR3 | LV- |
| LV-FR1 | L2) | LV-FR2 | (CDR2-L2) | LV-FR3 | (CDR3-L2) | FR4 |
| DIQMTQSPSSL | SASSSV | WYQQKP | DTSKLAS | GVPSRFSG | QQWSSN | FTFG |
| SASVGDRVTIT | SYMN | GKAPKRLI | SEQ ID NO | SGSGTDFTL | P SEQ ID | QGTK |
| C SEQ ID NO 33 | SEQ ID | Y SEQ ID | 84 | TISSLQPED | NO 151 | LEIK |
| | NO 83 | NO 150 | | FATYYC | | SEQ |
| | | | | SEQ ID NO | | ID NO |
| | | | | 35 | | 152 |
| DIQLTQSPAIM | RASSSV | WYQQKS | DTSKVAS | GVPYRFSG | QQWSSN | FGAG |
| SASPGEKVTM | SYMN | GTSPKRW | SEQ ID NO | SGSGTSYSL | PLT SEQ | TKLEL |
| TC SEQ ID NO | SEQ ID | IY SEQ | 81 | TISSMEAED | ID NO 82 | K SEQ |
| 102 | NO 80 | ID NO 103 | | AATYYC | | ID NO |
| | | | | SEQ ID NO | | 105 |
| | | | | 104 | | |
| QIVLTQSPAIM | SASSSV | WYQQKS | DTSKLAS | GVPAHFRG | QQWSSN | FGSG |
| SASPGEKVTM | SYMN | GTSPKRW | SEQ ID NO | SGSGTSYSL | PFT SEQ | TKLEI |
| TC SEQ ID NO | SEQ | IY SEQ | 84 | TISGMEAED | ID NO 85 | N SEQ |
| 107 | ID NO 83 | ID NO 103 | | AATYYC | | ID NO |
| | | | | SEQ ID NO | | 109 |
| | | | | 108 | | |
| DIQMTQSPSSL | SASSSV | WYQQTP | DTSKLAS | GVPSRFSG | QQWSSN | FGQG |
| SASVGDRVTIT | SYMN | GKAPKR | SEQ ID NO | SGSGTDYT | PFT SEQ | TKLQI |
| C SEQ ID NO | SEQ ID | WIY SEQ | 84 | FTISSLQPE | ID NO 85 | T SEQ |
| 114 | NO 83 | ID NO 115 | | DIATYYC | | ID NO |
| | | | | SEQ ID NO | | 117 |
| | | | | 116 | | |
| EIVLTQSPATL | RASQS | WYQQKP | DASNRAT | GIPARFSGS | QQRSNW | FGGG |
| SLSPGERATLS | VSSYLA | GQAPRLLI | SEQ ID NO | GSGTDFTLT | PPLT | TKVEI |
| C SEQ ID NO | SEQ ID | Y SEQ ID | 90 | ISSLEPEDF | SEQ ID NO | K SEQ |
| 122 | NO 89 | NO 123 | | AVYYC | 91 | ID NO |
| | | | | SEQ ID NO | | 125 |
| | | | | 124 | | |
| DIQLTQPNSVS | TLSSGN | WYQLYEG | DDDKRPD | GVPDRFSG | HSYVSSF | FGGG |
| TSLGSTVKLSC | IENNYV | RSPTTMIY | SEQ ID NO | SIDRSSNSA | NV | TKLTV |
| SEQ ID NO 130 | H SEQ | SEQ ID NO | 96 | FLTIHNVAIE | SEQ ID NO | L |
| | ID NO 95 | 131 | | DEAIYFC | 97 | SEQ |
| | | | | SEQ ID NO | | ID NO |
| | | | | 132 | | 133 |
| QTVVTQEPSLT | GSSTG | WVQQKP | GTKFLAP | GTPARFSG | VLWYSNR | FGGG |
| VSPGGTVTLT | AVTSG | GQAPRGL | SEQ ID NO | SLLGGKAAL | WV | TKLTV |
| C | NYPN | IG | 179 | TLSGVQPE | SEQ ID NO | L |
| SEQ ID NO 184 | SEQ ID | SEQ ID NO | | DEAEYYC | 180 | SEQ |
| | NO 178 | 185 | | SEQ ID NO | | ID NO |
| | | | | 186 | | 133 |
| QAVVTQEPSL | GSSTG | WVQEKP | GTNKRAP | GTPARFSG | ALWYSNL | FGGG |
| TVSPGGTVTLT | AVTTSN | GQAFRGL | SEQ ID NO | SLLGGKAAL | WV | TKLTV |
| C | YAN | IG | 182 | TLSGAQPE | SEQ ID NO | L |
| SEQ ID NO 187 | SEQ ID | SEQ ID NO | | DEAEYYC | 183 | SEQ |
| | NO 181 | 188 | | SEQ ID NO | | ID NO |
| | | | | 189 | | 133 |

The variable heavy chain sequence of the anti-oxMIF antibody can be as follows:

(SEQ ID NO 158)
EVQLLESGGGLVQPGGSLRLSCAASGFTFSIYSMNWVRQAPGKGLEWVSS
IGSSGGTTYYADSVKGRFTISRDNSKNTLYLQMNSLRAEDTAVYYCAGSQ
WLYGMDVWGQGTTVTVSS.

The variable light chain sequence of the anti-oxMIF antibody can be as follows:

(SEQ ID NO 134)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIK.

The variable heavy chain sequence of the anti-CD3 antibody can be as follows:

(SEQ ID NO 135)
QVQLVQSGAEVKKPGASVKVSCKASGYTFTRYTMHWRQAPGQGLEWMGYI
NPSRGYTNYNQKFKDRVTLTTDKSSSTAYMELSSLRSEDTAVYYCARYYD
DHYSLDYWGQGTLVTVSS.

The variable light chain sequence of the anti-CD3 antibody can be as follows:

(SEQ ID NO 136)
DIQMTQSPSSLSASVGDRVTITCSASSSVSYMNWYQQKPGKAPKRLIYDT
SKLASGVPSRFSGSGSGTDFTLTISSLQPEDFATYYCQQWSSNPFTFGQG
TKLEIK.

The variable heavy chain sequence of the anti-CD3 antibody can also be as follows:

(SEQ ID NO 190)
EVQLVESGGGLVQPGGSLKLSCAASGFTFNKYAMNWRQAPGKGLEWVARI
RSKYNNYATYYADSVKDRFTISRDDSKNTAYLQMNNLKTEDTAVYYCVRH
GNFGNSYISYWAYWGQGTLVTVSS.

The variable light chain sequence of the anti-CD3 antibody can further be as follows

(SEQ ID NO 191)
QTVVTQEPSLTVSPGGTVTLTCGSSTGAVTSGNYPNWQQKPGQAPRGLIG
GTKFLAPGTPARFSGSLLGGKAALTLSGVQPEDEAEYYCVLWYSNRWVFG
GGTKLTVL.

In a further alternative embodiment, the variable heavy chain sequence of the anti-CD3 antibody can also be as follows:

(SEQ ID NO 192)
EVQLLESGGGLVQPGGSLRLSCAASGFTFSTYAMNWVRQAPGKGLEWVSR
IRSKYNNYATYYADSVKGRFTISRDDSKNTLYLQMNSLRAEDTAVYYCVR
HGNFGNSYVSWFAYWGQGTLVTVSS.

In a further alternative embodiment, the variable light chain sequence of the anti-CD3 antibody can be as follows

(SEQ ID NO 193)
QAVVTQEPSLTVSPGGTVTLTCGSSTGAVTTSNYANVQEKPGQAFRGLIG
GTNKRAPGTPARFSGSLLGGKAALTLSGAQPEDEAEYYCALWYSNLWVFG
GGTKLTVL.

Specifically, the ε chain of CD3 can comprise the sequence

(SEQ ID NO 141)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI.

Specifically, the δ chain of CD3 can comprise the sequence:

(SEQ ID NO 142)
MEHSTFLSGLVLATLLSQVSPFKIPIEELEDRVFVNCNTSITVEGTVGTL
LSDITRLDLGKRILDPRGIYRCNGTDIYKDKESTVQVHYRMCQSCVELDP
ATVAGIIVTDVIATLLLALGVFCFAGHETGRLSGAADTQALLRNDQVYQP
LRDRDDAQYSHLGGNWARNK.

Specifically, the γ chain of CD3 can comprise the sequence

(SEQ ID NO 143)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

According to a specific embodiment, the domain of oxMIF specifically recognized by the oxMIF binding site comprises the sequence

(SEQ ID NO 144)
MPMFIVNTNVPRASVPDGFLSELTQQLAQATGKPPQYIAVHVVPDQLMAF
GGSSEPCALCSLHSIGKIGGAQNRSYSKLLCGLLAERLRISPDRVYINYY
DMNAANVGWNNSTFA.

Specifically, any one of SEQ ID Nos 134 to SEQ ID NO 144 and SEQ ID NO 158 can comprise 1, 2, 3, or 4 point mutations.

A “point mutation” is particularly understood as the engineering of a polynucleotide that results in the expression of an amino acid sequence that differs from the non-engineered amino acid sequence in the substitution or exchange, deletion or insertion of one or more single or doublets of amino acids for different amino acids. Preferred point mutations refer to the exchange of amino acids of the same polarity and/or charge. In this regard, amino acids refer to twenty naturally occurring amino acids encoded by sixty-one triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) sequence identity” with respect to the polypeptide sequences identified herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

According to the present invention, sequence identity of the CDR or framework region sequences is at least 70%, 75%, 80%, 85%, 90%, 95%, 96%, 97%, 98%, 99%, 99.5% or 100% with the respective sequences described herein.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-oxMIF/anti-CD3 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“No substantial cross-reactivity” means that a molecule (e.g., an antibody) does not recognize or specifically bind an antigen different from the actual target antigen of the molecule (e.g. an antigen closely related to the target antigen), specifically reduced MIF, particularly when compared to that target antigen. For example, an antibody may bind less than about 10% to less than about 5% to an antigen different from the actual target antigen, or may bind said antigen different from the actual target antigen at an amount consisting of less than about 10%, 9%, 8% 7%, 6%, 5%, 4%, 3%, 2%, 1%, 0.5%, 0.2%, or 0.1%, preferably less than about 2%, 1%, or 0.5%, and most preferably less than about 0.2% or 0.1% antigen different from the actual target antigen. Binding can be determined by any method known in the art such as, but not limited to ELISA or surface plasmon resonance.

The recombinant production of the antibody of the invention preferably employs an expression system, e.g. including expression constructs or vectors comprising a nucleotide sequence encoding the antibody format.

The term “expression system” refers to nucleic acid molecules containing a desired coding sequence and control sequences in operable linkage, so that hosts transformed or transfected with these sequences are capable of producing the encoded proteins. In order to effect transformation, the expression system may be included on a vector; however, the relevant DNA may then also be integrated into the host chromosome. Alternatively, an expression system can be used for in vitro transcription/translation.

“Expression vectors” used herein are defined as DNA sequences that are required for the transcription of cloned recombinant nucleotide sequences, i.e. of recombinant genes and the translation of their mRNA in a suitable host organism. Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences.

Specifically, the term refers to a vehicle by which a DNA or RNA sequence (e.g. a foreign gene), e.g. a nucleotide sequence encoding the antibody format of the present invention, can be introduced into a host cell, so as to transform the host and promote expression (e.g. transcription and translation) of the introduced sequence. Plasmids are preferred vectors of the invention.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites.

A “cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA that can readily accept additional (foreign) DNA and which can readily be introduced into a suitable host cell. A vector of the invention often contains coding DNA and expression control sequences, e.g. promoter DNA, and has one or more restriction sites suitable for inserting foreign DNA. Coding DNA is a DNA sequence that encodes a particular amino acid sequence for a particular polypeptide or protein such as an antibody format of the invention. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors of the invention will often include one or more replication systems for cloning or expression, one or more markers for selection in the host, e.g. antibiotic resistance, and one or more expression cassettes.

The procedures used to ligate DNA sequences, e.g. providing or coding for the factors of the present invention and/or the protein of interest, a promoter, a terminator and further sequences, respectively, and to insert them into suitable vectors containing the information necessary for integration or host replication, are well known to persons skilled in the art, e.g. described by J. Sambrook et al., “Molecular Cloning 2nd ed.”, Cold Spring Harbor Laboratory Press (1989).

A host cell is specifically understood as a cell, a recombinant cell or cell line transfected with an expression construct, such as a vector according to the invention.

The term “host cell line” as used herein refers to an established clone of a particular cell type that has acquired the ability to proliferate over a prolonged period of time. The term host cell line refers to a cell line as used for expressing an endogenous or recombinant gene to produce polypeptides, such as the recombinant antibody format of the invention.

A “production host cell” or “production cell” is commonly understood to be a cell line or culture of cells ready-to-use for cultivation in a bioreactor to obtain the product of a production process, the recombinant antibody format of the invention. The host cell type according to the present invention may be any prokaryotic or eukaryotic cell.

The term “recombinant” as used herein shall mean “being prepared by genetic engineering” or “the result of genetic engineering”, e.g. specifically employing heterologous sequences incorporated in a recombinant vector or recombinant host cell.

A bispecific antibody of the invention may be produced using any known and well-established expression system and recombinant cell culturing technology, for example, by expression in bacterial hosts (prokaryotic systems), or eukaryotic systems such as yeasts, fungi, insect cells or mammalian cells. An antibody molecule of the present invention may be produced in transgenic organisms such as a goat, a plant or a transgenic mouse, an engineered mouse strain that has large fragments of the human immunoglobulin loci and is deficient in mouse antibody production. An antibody may also be produced by chemical synthesis.

According to a specific embodiment, the host cell is a production cell line of cells selected from the group consisting of CHO, PerC6, CAP, HEK, HeLa, NS0, SP2/0, hybridoma and Jurkat. More specifically, the host cell is obtained from CHO cells.

The host cell of the invention is specifically cultivated or maintained in a serum-free culture, e.g. comprising other components, such as plasma proteins, hormones, and growth factors, as an alternative to serum.

Host cells are most preferred, when being established, adapted, and completely cultivated under serum free conditions, and optionally in media which are free of any protein/peptide of animal origin.

Anti-oxMIF/anti-CD3 antibodies can be recovered from the culture medium using standard protein purification methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

As used herein, “treatment”, “treat” or “treating” refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The anti-oxMIF/anti-CD3 antibody of the invention and the pharmaceutical compositions comprising it, can be administered in combination with one or more other therapeutic, diagnostic or prophylactic agents. Additional therapeutic agents include other anti-neoplastic, antitumor, anti-angiogenic, chemotherapeutic agents, steroids, or checkpoint inhibitors depending on the disease to be treated.

The pharmaceutical compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. As will be appreciated by the skilled artisan, the route and/or mode of administration will vary depending upon the desired results.

The anti-oxMIF/anti-CD3 antibody may be administered once, but more preferably is administered multiple times. For example, the antibody may be administered from three times daily to once every six months or longer. The administering may be on a schedule such as three times daily, twice daily, once daily, once every two days, once every three days, once weekly, once every two weeks, once every month, once every two months, once every three months and once every six months.

The term “cancer” as used herein refers to proliferative diseases, specifically to solid cancers, such as colorectal cancer, ovarian cancer, pancreas cancer, lung cancer, melanoma, squamous cell carcinoma (SCC) (e.g., head and neck, esophageal, and oral cavity), hepatocellular carcinoma, colorectal adenocarcinoma, kidney cancer, medullary thyroid cancer, papillary thyroid cancer, astrocytic tumor, neuroblastoma, Ewing's sarcoma, cervical cancer, endometrial carcinoma, breast cancer, prostate cancer, and malignant seminoma, including refractory versions of any of the above cancers, or a combination of one or more of the above cancers.

Detection of cellular expression of oxMIF can be performed with the antibody as described herein, said antibody being labeled so that specific expression of oxMIF can be detected. Antibody labelling can be performed according to methods well known in the art. Such labels can be, but are not limited to radioisotopes, fluorescent labels, chemiluminescent labels, enzyme labels, and bioluminescent labels.

The invention further encompasses following items:

1. An anti-oxMIF/anti-CD3 antibody or antigen binding fragment thereof, selected from the group consisting of

an IgG wherein a scFv is fused to only one of the two heavy chains,

an IgG wherein one Fab arm is replaced by a bispecific-T-cell-engager (BiTE), and

an IgG wherein both Fab arms are replaced by scFvs with different binding specificities,

comprising at least one binding site specifically recognizing oxMIF and one binding site specifically recognizing CD3,

and wherein the site specifically recognizing oxMIF comprises

(a) a heavy chain variable region comprising

a CDR1-H1 sequence which has at least 70% sequence identity to any of the sequences selected from the group consisting of SEQ ID NO 1, SEQ ID NO 7, SEQ ID NO 13, SEQ ID NO 19 and SEQ ID NO 26, and

a CDR2-H1 sequence which has at least 70% sequence identity to any of the sequences selected from the group consisting of SEQ ID NO 2, SEQ ID NO 8, SEQ ID NO 14, SEQ ID NO 20 and SEQ ID NO 27, and

a CDR3-H1 sequence which has at least 70% sequence identity to any of the sequences selected from the group consisting of SEQ ID NO 3, SEQ ID NO 9, SEQ ID NO 15 and SEQ ID NO 21, and

(b) a light chain variable region comprising

a CDR1-L1 sequence which has at least 70% sequence identity to any of the sequences selected from the group consisting of SEQ ID NO 4, SEQ ID NO 10, SEQ ID NO 16, SEQ ID NO 22, SEQ ID NO 28 and SEQ ID NO 138, and

a CDR2-L1 sequence which has at least 70% sequence identity to any of the sequences selected from the group consisting of SEQ ID NO 5, SEQ ID NO 11, SEQ ID NO 17, SEQ ID NO 23 and SEQ ID NO 25, and

a CDR3-L1 sequence which has at least 70% sequence identity to any of the sequences selected from the group consisting of SEQ ID NO 6, SEQ ID NO 12, SEQ ID NO 18 and SEQ ID NO 24.

2. The anti-oxMIF/anti-CD3 antibody of item 1, wherein the IgG is recognizing oxMIF and the scFv is recognizing CD3, further comprising peptide linkers joining the CD3 variable light (VL) and variable heavy (VH) chains.

3. The anti-oxMIF/anti-CD3 antibody of item 1, wherein the IgG Fab arm is recognizing oxMIF and the bispecific T-cell engager is recognizing oxMIF and CD3, further comprising peptide linkers joining the VL and VH chains.

4. The anti-oxMIF/anti-CD3 antibody of item 1, wherein both Fab arms are replaced by scFvs and wherein one scFv is targeting oxMIF and the other scFv is targeting CD3, further comprising peptide linkers joining the VL and VH chains.

5. The anti-oxMIF/anti-CD3 antibody of any one of items 1 to 4, comprising 0, 1, or 2 point mutations in each of the CDR sequences which are the

CDR1-H1 sequence selected from the group consisting of SEQ ID NO 1, SEQ ID NO 7, SEQ ID NO 13, SEQ ID NO 19 and SEQ ID NO 26, and

CDR2-H1 sequence selected from the group consisting of SEQ ID NO 2, SEQ ID NO 8, SEQ ID NO 14, SEQ ID NO 20 and SEQ ID NO 27, and

CDR3-H1 sequence selected from the group consisting of SEQ ID NO 3, SEQ ID NO 9, SEQ ID NO 15 and SEQ ID NO 21, and

CDR1-L1 sequence selected from the group consisting of SEQ ID NO 4, SEQ ID NO 10, SEQ ID NO 16, SEQ ID NO 22, SEQ ID NO 28, and SEQ ID NO 138, and

CDR2-L1 sequence selected from the group consisting of SEQ ID NO 5, SEQ ID NO 11, SEQ ID NO 17, SEQ ID NO 23 and SEQ ID NO 25, and

CDR3-L1 sequence selected from the group consisting of SEQ ID NO 6, SEQ ID NO 12, SEQ ID NO 18, SEQ ID NO 24, and SEQ ID NO 153.

6. The anti-oxMIF/anti-CD3 antibody according to any one of items 1 to 4, wherein the binding site specifically recognizing CD3 comprises

(a) a heavy chain variable region comprising a CDR1-H2 sequence which has at least 70% sequence identity to any of the sequences selected from the group consisting of SEQ ID NO 77, SEQ ID NO 86 and SEQ ID NO 92, and

a CDR2-H2 which has at least 70% sequence identity to any of the sequences selected from the group consisting of SEQ ID NO 78, SEQ ID NO 87, and SEQ ID NO 93, and

a CDR3-H2 which has at least 70% sequence identity to any of the sequences selected from the group consisting of SEQ ID NO 79, SEQ ID NO 88, SEQ ID NO 94, and SEQ ID NO 149, and
(b) a light chain comprising

a CDR1-L2 which has at least 70% sequence identity to any of the sequences selected from the group consisting of SEQ ID NO 80, SEQ ID NO 83, SEQ ID NO 89 and SEQ ID NO 95, and

a CDR2-L2 which has at least 70% sequence identity to any of the sequences selected from the group consisting of SEQ ID NO 81, SEQ ID NO 84, SEQ ID NO 90 and SEQ ID NO 96, and

a CDR3-L2 which has at least 70% sequence identity to any of the sequences selected from the group consisting of SEQ ID NO 82, SEQ ID NO 85, SEQ ID NO 91, SEQ ID NO 97, and SEQ ID NO 151.

7. The anti-oxMIF/anti-CD3 antibody according to any one of items 1 to 6, comprising 0, 1, or 2 point mutations in each of the CDR sequences which are the

CDR1-H2 sequence selected from the group consisting of SEQ ID NO 77, SEQ ID NO 86 and SEQ ID NO 92, and

CDR2-H2 sequence selected from the group consisting of SEQ ID NO 78, SEQ ID NO 87, and SEQ ID NO 93, and

CDR3-H2 sequence selected from the group consisting of SEQ ID NO 79, SEQ ID NO 88, SEQ ID NO 94, and SEQ ID NO 149, and

CDR1-L2 sequence selected from the group consisting of SEQ ID NO 80, SEQ ID NO 83, SEQ ID NO 89 and SEQ ID NO 95, and

CDR2-L2 sequence selected from the group consisting of SEQ ID NO 81, SEQ ID NO 84, SEQ ID NO 90 and SEQ ID NO 96, and

CDR3-L2 sequence selected from the group consisting of SEQ ID NO 82, SEQ ID NO 85, SEQ ID NO 91, SEQ ID NO 97, and SEQ ID NO 151.

8. The anti-oxMIF/anti-CD3 antibody according to any one of items 1 to 7, comprising the sequences SEQ ID NO 7, SEQ ID NO 8, SEQ ID NO 9, SEQ ID NO 10, SEQ ID NO 11, SEQ ID NO 12, SEQ ID NO 77, SEQ ID NO 78, SEQ ID NO 149, SEQ ID NO 83, SEQ ID NO 84, and SEQ ID NO 151.

9. The anti-oxMIF/anti-CD3 antibody according to any one of items 1 to 8, wherein the binding site specifically recognizing oxMIF comprises a heavy chain variable region having at least 70%, preferably at least 80%, preferably at least 90%, more preferably at least 95%, more preferably at least 99,5% sequence identity to the amino acid sequence of SEQ ID NO 158, and a light chain variable region having at least 70%, preferably at least 80%, preferably at least 90%, more preferably at least 95% sequence, more preferably at least 99,5% identity to the amino acid sequence of SEQ ID NO 134.

10. The anti-oxMIF/anti-CD3 antibody according to any one of items 1 to 9, wherein the binding site specifically recognizing CD3 comprises a heavy chain variable region having at least 70%, preferably at least 80%, preferably at least 90%, more preferably at least 95% sequence identity to the amino acid sequence of SEQ ID NO 135 and a light chain variable region having at least 70%, preferably at least 80%, preferably at least 90%, more preferably at least 95% sequence identity to the amino acid sequence of SEQ ID NO 136.

11. The anti-oxMIF/anti-CD3 antibody according to any one of items 1 to 10, comprising the amino acid sequence of SEQ ID NO 159, SEQ ID NO 137, SEQ ID NO 140, SEQ ID NO 160, SEQ ID NO 161, SEQ ID NO 162, SEQ ID NO 163 or an amino acid sequence having at least 85%, 90%, specifically at least 95%, specifically at least 99% sequence identity with any one of SEQ ID NO 159, SEQ ID NO 137, SEQ ID NO 140, SEQ ID NO 160, SEQ ID NO 161, SEQ ID NO 162, SEQ ID NO 163.

12. A pharmaceutical composition comprising the anti-oxMIF/anti-CD3 antibody of items 1 to 11 and a pharmaceutically acceptable carrier or excipient.

13. The anti-oxMIF/anti-CD3 antibody according to any one of items 1 to 11 or the pharmaceutical composition of claim 12 for use in the treatment of cancer, specifically in the treatment of colorectal cancer, ovarian cancer, pancreas cancer, lung cancer.

14. The anti oxMIF/anti-CD3 antibody according to any one of items 1 to 11 for use as a medicament.

15. Isolated nucleic acid molecule(s) encoding an anti oxMIF/anti-CD3 antibody according to any one of items 1 to 11.

16. An expression vector comprising nucleic acid molecule(s) of item 15.

17. A host cell comprising a vector according to item 18.

18. A method of producing the anti-oxMIF/anti-CD3 antibody according to any one of items 1 to 11, comprising expressing a nucleic acid encoding the antibody in a host cell.

19. An in vitro method of detecting cellular expression of oxMIF, the method comprising: contacting a biological sample comprising a human cell to be tested with an anti-oxMIF/anti-CD3 antibody according to any one of items 1 to 11; and detecting binding of said antibody; wherein the binding of said antibody indicates the presence of oxMIF on the cell, to thereby detect whether the cell expresses oxMIF.

20. The in vitro method of item 21, wherein the biological sample comprises intact human cells, tissues, biopsy probes, or a membrane fraction of a cell of interest.

21. The in vitro method of item 19 or 20, wherein the anti-oxMIF/anti-CD3 antibody is labeled with a detectable label selected from the group consisting of a radioisotope, a fluorescent label, a chemiluminescent label, an enzyme label, and a bioluminescent label.

22. The anti-oxMIF/anti-CD3 antibody of items 1 to 11 for use in diagnosing a cancer expressing oxMIF in a subject, wherein said antibody is conjugated to a detectable label.

The foregoing description will be more fully understood with reference to the following examples. Such examples are, however, merely representative of methods of practicing one or more embodiments of the present invention and should not be read as limiting the scope of invention.

### EXAMPLES

### Example 1

Biochemical characterization of bispecific antibodies

The anti-oxMIF/anti-CD3 antibodies are tested as described below to ensure quality and functionality.

1) Identity: Method: by Electrospray ionization MS (ESI-MS)

2) Molecular integrity: Method: SEC multi-angle light scattering (SEC MALS)

3) Purity: Method: SDS PAGE

4) Binding and affinity: Methods: ELISA, Biacore, FACS as described below

ELISA according to Thiele M. et al., 2015, J Immunol 2015; 195:2343-2352: For determination of oxMIF specificity, anti-oxMIF/anti-CD3 antibodies are coated into microplates and incubated with recombinant MIF (control), oxMIF, or oxMIF reduced with DTT (control). Captured MIF or oxMIF is detected with rabbit anti-MIF Abs and a goat anti-rabbit-IgG-HRP conjugate. Plates are stained with 3,3′,5,5′-Tetramethylbenzidine. For determination of CD3 specificity, anti-oxMIF/anti-CD3 antibodies are coated into microplates and incubated with recombinant Human CD3 epsilon protein. Captured CD3 is detected with rabbit anti-CD3 Abs and a goat anti-rabbit-IgG-HRP conjugate. Plates are stained with 3,3′,5,5′-Tetramethylbenzidine.

SPR (Biacore) according to Hoellriegl et al., Eur J Pharmacol. 2018 Feb. 5; 820:206-216: Binding affinities and kinetic constants of anti-oxMIF/anti-CD3 (anti-oxMIF/CD3) bispecific antibodies are determined by surface plasmon resonance using either an antibody-capture format (anti-oxMIF/CD3 bispecific abs captured on sensor chip) or an antigen-capture format (recombinant MIF or recombinant CD3 (epsilon, delta or gamma chain) captured on a sensor chip). Measurements are conducted on a T200 Biacore instrument.

Specifically, anti-oxMIF/anti-CD3 antibody or a non-binding control antibody is immobilized to Biacore CM5 optical sensor chips (GE Healthcare, Piscataway, N.J.) using standard amine coupling conditions. Recombinant MIF is diluted in HBS-EP buffer (GE Healthcare) to concentrations of 50, 75, 100, or 150 nM in the presence of 0.2% Proclin300 (active component 5-chloro-2-methyl-4-isothiazolin-3-one; Sigma) to transform MIF into an oxMIF surrogate (Thiele M. et al., 2015, J Immunol 2015; 195:2343-2352). Proclin300 treated MIF is applied to immobilized anti-oxMIF/anti-CD3 antibody and affinity measured with a Biacore™ 3000 Instrument (GE Healthcare). The kinetics of the concentration series are analyzed by local simultaneous association/dissociation fitting of each binding curve to the iterative Langmuir 1:1 interaction model with mass transfer compensation provided by the BiaEvaluation software (GE Healthcare).

FACS: oxMIF positive cancer cells (e.g. PC3 or A2780) are incubated with anti-oxMIF/anti-CD3 bispecific abs or controls. Unlabeled Abs are detected by R-PE-labeled goat anti-human IgG Ab (from Sigma). Data are acquired on a FACS Canto II (BD Biosciences).

### Example 2

Biodistribution and PK study

Biodistribution and pharmacokinetics (PK) of the anti-oxMIF/anti-CD3 antibodies are determined by PET-imaging. The bispecific anti-oxMIF/anti-CD3 antibodies are labelled and pharmacokinetics of the proteins in the tumor, circulation and major organs are determined in SCID mice bearing a subcutaneous SKOV-3 tumor or another appropriate cell line.

Exploratory PD Study

1) Xenograft NOD/SCID SKOV-3 model: A dose response curve of the anti-oxMIF/anti-CD3 bispecific antibodies is determined in a NOD/SCID SKOV-3 xenograft mouse model for ovarian cancer applying human lymphocytes (Xing, J., et al., Translational Oncology (2017) 10, 780-785)

Briefly, fresh cultured SKOV-3 cells (1×10⁶) are mixed with fresh isolated human PBMCs (5×10⁶) in 200-μl volume and subcutaneously co-implanted into the right flank of 5-week-old male NOD/SCID mice. Two hours after tumor cell injection, mice are treated with anti-oxMIF/anti-CD3 antibodies every 3 days by intraperitoneal injection. The anti-oxMIF/anti-CD3 bispecific antibodies are applied in 6 doses, the respective control bispecific antibodies in the highest dose. Mice are weighed and tumor growth is measured twice a week using calipers. Tumor volume is calculated as 1/2(length×width²).

As an alternative, PD of anti-oxMIF/anti-CD3 antibodies is monitored by bioluminescence. Briefly, thirty 5-weeks old NSG mice (The Jackson Laboratory) are each given 1×10⁶ IGROV1-ffluc intraperitoneally (i.p.) on day 0. On day 2, the animals are i.p. injected with 150 mg/kg D-luciferin (15 mg/mL stock solution; Biosynth) and divided into 5 groups of 6 animals each by average bioluminescence. On day 6, each animal (except the no treatment cohort) is i.p. injected with 1×10⁷ primary T cells expanded from healthy donor PBMC, and 1 h later, with anti-oxMIF/CD3 antibodies in 4 different doses or PBS alone. This is repeated for a total of 10 daily (day 6 to 15) i.p. injections. Every 3-4 days, tumor growth is monitored by bioluminescent imaging 5 min after i.p. injections with 150 mg/kg D-luciferin. The weight of the mice is measured every 1-4 days.

2) Primary ovarian human xenograft model: The anti-oxMIF/anti-CD3 bispecific antibodies are tested essentially as described in Schleret B. et al., Cancer Res 2005; 65(7): 2882-9.

In brief, following surgical resection of peritoneal metastasis of histologically proven ovarian cancer patients, primary tumor specimens are cut into 50 to 100 mm3 cubes and s.c. implanted into NOD/SCID mice. Animals are i.v. treated with anti-oxMIF/anti-CD3 bispecific antibody formats or control antibody. The anti-oxMIF/anti-CD3 bispecific antibodies are applied in 3 doses, the respective control in the highest dose. Tumor sizes are measured twice a week with a caliper in two perpendicular dimensions and tumor volumes calculated according to tumor volume=[(width²×length)/2].

As an alternative: 1×10⁶ human PBMCs isolated from heparinized fresh whole blood of a healthy donor are mixed with 5×10⁵ primary tumor-initiating cells (TICs) in a final volume of 200 μl. The PBMC effector/target cell mixture (E:T of 2:1) is s.c. injected into the right flank of each NOD/SCID mouse. The mice are intravenously treated with anti-oxMIF/CD3 antibodies or PBS control vehicle starting 2 h after inoculation with 3 different doses.

For elimination of established tumors in NOD/SCID mice by treatment with anti-oxMIF/CD3 antibodies, mixtures of 5×10⁶ TICs and 1×10⁷ human PBMCs are inoculated into 5 NOD/SCID mice per group to allow solid tumor formation. After tumor establishment at day 4, mice are treated i.v. for 14 days with three different doses of anti-oxMIF/CD3 antibodies, or with vehicle control in presence of PBMCs.

### Example 3

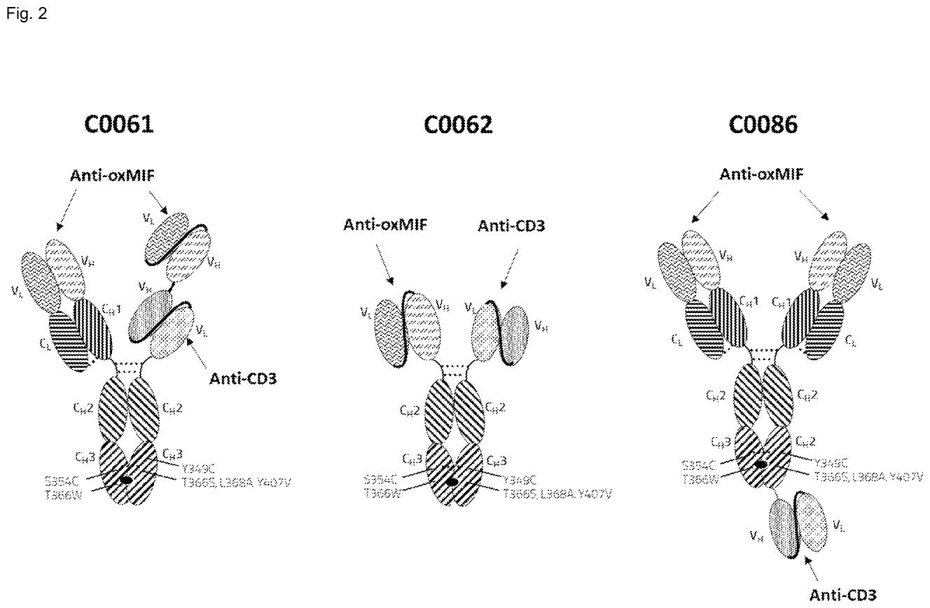

Overview on the antibodies used in the examples. The respective formats are schematically depicted in FIG. 2.

C0061 (Anti-oxMIF Fab and anti-oxMIF/anti-CD3 BiTE fused to Fc; Fab-BiTE-Fc):

Polypeptide 1:
(SEQ ID NO 159)
EVQLLESGGGLVQPGGSLRLSCAASGFTFSIYSMNWRQAPGKGLEWWSSI
GSSGGTTYYADSVKGRFTISRDNSKNTLYLQMNSLRAEDTAVYYCAGSQW
LYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RWVSVLTVLHQDWLNGKEYKCKVSNKALPAPIEKTISKAKGQPREPQVYT
LPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGK
Polypeptide 2:
(SEQ ID NO 137)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKGGGGSGGGGSGGGGSEVQLLESGGGLVQPGGSLRLSCAASGFT
FSIYSMNWVRQAPGKGLEWVSSIGSSGGTTYYADSVKGRFTISRDNSKNT
LYLQMNSLRAEDTAVYYCAGSQWLYGMDVWGQGTTVTVSSGGGGSQVQLV
QSGAEVKKPGASVKVSCKASGYTFTRYTMHWVRQAPGQGLEWMGYINPSR
GYTNYNQKFKDRVTLTTDKSSSTAYMELSSLRSEDTAVYYCARYYDDHYS
LDYWGQGTLVTVSSGGSGGSGGSGGSGGSDIQMTQSPSSLSASVGDRVTI
TCSASSSVSYMNWYQQKPGKAPKRLIYDTSKLASGVPSRFSGSGSGTDFT
LTISSLQPEDFATYYCQQWSSNPFTFGQGTKLEIKGGGGSDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSPGK
Polypeptide 3:
(SEQ ID NO 140)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

C0062 (anti-oxMIF scFv and anti-CD3 scFv fused to Fc; scFv(oxMIF)-scFv(CD3)-Fc; (scFv)₂-Fc):

Polypeptide 1:
(SEQ ID NO 160)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKGGGGSGGGGSGGGGSEVQLLESGGGLVQPGGSLRLSCAASGFT
FSIYSMNWRQAPGKGLEWSSIGSSGGTTYYADSVKGRFTISRDNSKNTLY
LQMNSLRAEDTAVYYCAGSQWLYGMDVWGQGTTVTVSSGGGGSDKTHTCP
PCPAPELLGGPSVFLFPPKPKDTLMISRTPEVTCVWDVSHEDPEVKFNWY
VDGVEVHNAKTKPREEQYNSTYRVVSVLTVLHQDWLNGKEYKCKVSNKAL
PAPIEKTISKAKGQPREPQVYTLPPCRDELTKNQVSLWCLVKGFYPSDIA
VEWESNGQPENNYKTTPPVLDSDGSFFLYSKLTVDKSRWQQGNVFSCSVM
HEALHNHYTQKSLSLSPGKASAWSHPQFEK
Polypeptide 2:
(SEQ ID NO 161)
QVQLVQSGAEVKKPGASVKVSCKASGYTFTRYTMHWVRQAPGQGLEWMGY
INPSRGYTNYNQKFKDRVTLTTDKSSSTAYMELSSLRSEDTAVYYCARYY
DDHYSLDYWGQGTLVTVSSGGSGGSGGSGGSGGSDIQMTQSPSSLSASVG
DRVTITCSASSSVSYMNWYQQKPGKAPKRLIYDTSKLASGVPSRFSGSGS
GTDFTLTISSLQPEDFATYYCQQWSSNPFTFGQGTKLEIKGGGGSDKTHT
CPPCPAPELLGGPSVFLFPPKPKDTLMISRTPEVTCVVDVSHEDPEVKFN
WYVDGVEVHNAKTKPREEQYNSTYRVVSVLTVLHQDWLNGKEYKCKVSNK
ALPAPIEKTISKAKGQPREPQVCTLPPSRDELTKNQVSLSCAVKGFYPSD
IAVEWESNGQPENNYKTTPPVLDSDGSFFLVSKLTVDKSRWQQGNVFSCS
VMHEALHNHYTQKSLSLSPGKAAAHHHHHH

C0086 (Full anti-oxMIF IgG with one single anti-CD3 scFv fused to heavy chain; IgG1-scFv fusion; IgG-scFv)

Polypeptide 1:
(SEQ ID NO 162)
EVQLLESGGGLVQPGGSLRLSCAASGFTFSIYSMNWRQAPGKGLEWWSSI
GSSGGTTYYADSVKGRFTISRDNSKNTLYLQMNSLRAEDTAVYYCAGSQW
LYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RWVSVLTVLHQDWLNGKEYKCKVSNKALPAPIEKTISKAKGQPREPQVCT
LPPSRDELTKNQVSLSCAVKGFYPSDIAVEWESNGQPENNYKTTPPVLDS
DGSFFLVSKLTVDKSRWQQGNVFSCSVMHEALHNHYTQKSLSLSPGKGGG
GSGGGGSGGGGSQVQLVQSGAEVKKPGASVKVSCKASGYTFTRYTMHWRQ
APGQGLEWMGYINPSRGYTNYNQKFKDRVTLTTDKSSSTAYMELSSLRSE
DTAVYYCARYYDDHYSLDYWGQGTLVTVSSGGSGGSGGSGGSGGSDIQMT
QSPSSLSASVGDRVTITCSASSSVSYMNWYQQKPGKAPKRLIYDTSKLAS
GVPSRFSGSGSGTDFTLTISSLQPEDFATYYCQQWSSNPFTFGQGTKLEI
K
Polypeptide 2:
(SEQ ID NO 163)
EVQLLESGGGLVQPGGSLRLSCAASGFTFSIYSMNWRQAPGKGLEWWSSI
GSSGGTTYYADSVKGRFTISRDNSKNTLYLQMNSLRAEDTAVYYCAGSQW
LYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RWSVLTVLHQDWLNGKEYKCKVSNKALPAPIEKTISKAKGQPREPQVYTL
PPCRDELTKNQVSLWCLVKGFYPSDIAVEWESNGQPENNYKTTPPVLDSD
GSFFLYSKLTVDKSRWQQGNVFSCSVMHEALHNHYTQKSLSLSPGK
Polypeptide 3:
(SEQ ID NO 140)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

C0107 (Anti-oxMIF Fab and anti-oxMIF/anti-CD3 BiTE fused to Fc; Fab-BiTE-Fc):

Polypeptide 1:
(SEQ ID NO 194)
EVQLLESGGGLVQPGGSLRLSCAASGFTFSWYAMDWRQAPGKGLEWSGIY
PSGGRTKYADSVKGRFTISRDNSKNTLYLQMNSLRAEDTAVYYCARVNVI
AVAGTGYYYYGMDVWGQGTTVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVWDVSHEDPEVKFNWYVDGVEVHNAKTKPRE
EQYNSTYRVVSVLTVLHQDWLNGKEYKCKVSNKALPAPIEKTISKAKGQP
REPQVYTLPPCRDELTKNQVSLWCLVKGFYPSDIAVEWESNGQPENNYKT
TPPVLDSDGSFFLYSKLTVDKSRWQQGNVFSCSVMHEALHNHYTQKSLSL
SPK
Polypeptide 2:
(SEQ ID NO 137)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKGGGGSGGGGSGGGGSEVQLLESGGGLVQPGGSLRLSCAASGFT
FSIYSMNWVRQAPGKGLEWVSSIGSSGGTTYYADSVKGRFTISRDNSKNT
LYLQMNSLRAEDTAVYYCAGSQWLYGMDVWGQGTTVTVSSGGGGSQVQLV
QSGAEVKKPGASVKVSCKASGYTFTRYTMHWVRQAPGQGLEWMGYINPSR
GYTNYNQKFKDRVTLTTDKSSSTAYMELSSLRSEDTAVYYCARYYDDHYS
LDYWGQGTLVTVSSGGSGGSGGSGGSGGSDIQMTQSPSSLSASVGDRVTI
TCSASSSVSYMNWYQQKPGKAPKRLIYDTSKLASGVPSRFSGSGSGTDFT
LTISSLQPEDFATYYCQQWSSNPFTFGQGTKLEIKGGGGSDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSPGK
Polypeptide 3:
(SEQ ID NO 195)
EIVLTQSPGTLSLSPGERATLSCRASQGVSSSSLAWYQQKPGQAPRLLIY
GTSSRATGIPDRFSGSGSGTDFTLTISRLEPEDFAVYYCQQYGRSLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

C0111 (Anti-oxMIF Fab and anti-oxMIF/anti-CD3 BiTE fused to Fc; Fab-BiTE-Fc):

Polypeptide 1:
(SEQ ID NO 196)
EVQLLESGGGLVQPGGSLRLSCAASGFTFSIYSMNWRQAPGKGLEWWSSI
GSSGGTTYYADSVKGRFTISRDNSKNTLYLQMNSLRAEDTAVYYCAGSQW
LYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDT
LMISRTPEVTCVVVDVSHEDPEVKFNWYVDGVEVHNAKTKPREEQYNSTY
RWVSVLTVLHQDWLNGKEYKCKVSNKALPAPIEKTISKAKGQPREPQVYT
LPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGK
Polypeptide 2:
(SEQ ID NO 197)
EVQLLESGGGLVQPGGSLRLSCAASGFTFSIYSMNWRQAPGKGLEWWSSI
GSSGGTTYYADSVKGRFTISRDNSKNTLYLQMNSLRAEDTAVYYCAGSQW
LYGMDVWGQGTTVTVSSGGGGSGGGGSGGGGSDIQMTQSPSSLSASVGDR
VTITCRSSQRIMTYLNWYQQKPGKAPKLLIFVASHSQSGVPSRFRGSGSE
TDFTLTISGLQPEDSATYYCQQSFWTPLTFGGGTKVEIKSGGGGSEVQLV
ESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFG
NSYISYWAYWGQGTLVTVSSGGGGSGGGGSGGGGSQTWTQEPSLTVSPGG
TVTLTCGSSTGAVTSGNYPNWVQQKPGQAPRGLIGGTKFLAPGTPARFSG
SLLGGKAALTLSGVQPEDEAEYYCVLWYSNRWVFGGGTKLTVLGGGGSDK
THTCPPCPAPEAAGGPSVFLFPPKPKDTLMISRTPEVTCVVVDVSHEDPE
VKFNWYVDGVEVHNAKTKPREEQYNSTYRWSVLTVLHQDWLNGKEYKCKV
SNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVF
SCSVMHEALHNHYTQKSLSLSPGK
Polypeptide 3
(SEQ ID NO 140)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### Example 4: oxMIF-CD3 Bridging ELISA

Recombinant human MIF was immobilized into microwell plates at 1 μg/ml in PBS (transforming MIF to oxMIF according to Thiele M. et al., 2015, J Immunol 2015; 195:2343-2352). After blocking, bispecific antibodies were added to the plates at a concentration of 4 μg/ml. A dilution series of a FLAG-taggedCD3-ε-δ-Fc fusion protein was added and bound CD3 was detected using a monoclonal mouse anti-FLAG tag-HRP conjugate. OD was measured at 450 nM.

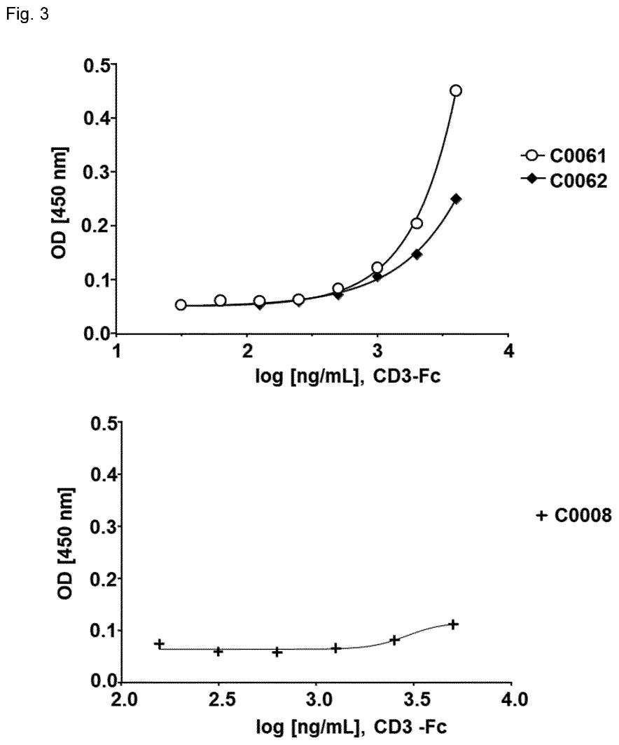

Simultaneous binding of bispecific antibodies C0061 and C0062 to oxMIF andCD3 is shown in FIG. 3.

FIG. 3 shows the simultaneous binding of anti-oxMIF bispecific antibodies C0061 and C0062 to oxMIF and CD3. The anti-oxMIF monospecific antibody C0008 was used as negative control.

### Example 5: Binding to oxMIF (ELISA)

Recombinant human MIF (1 μg/ml) diluted in PBS was immobilized into microwell plates (transforming MIF to oxMIF according to Thiele M. et al., 2015, J Immunol 2015; 195:2343-2352). After blocking, the bispecific antibodies were added to the plates at different concentrations. Bound bispecific antibodies were detected using protein L-HRP conjugate. Plates were developed by adding 3 3′5 5′tetramethylbenzidine (TMB) and chromogenic reaction was stopped with H2504. OD was measured at 450 nM.

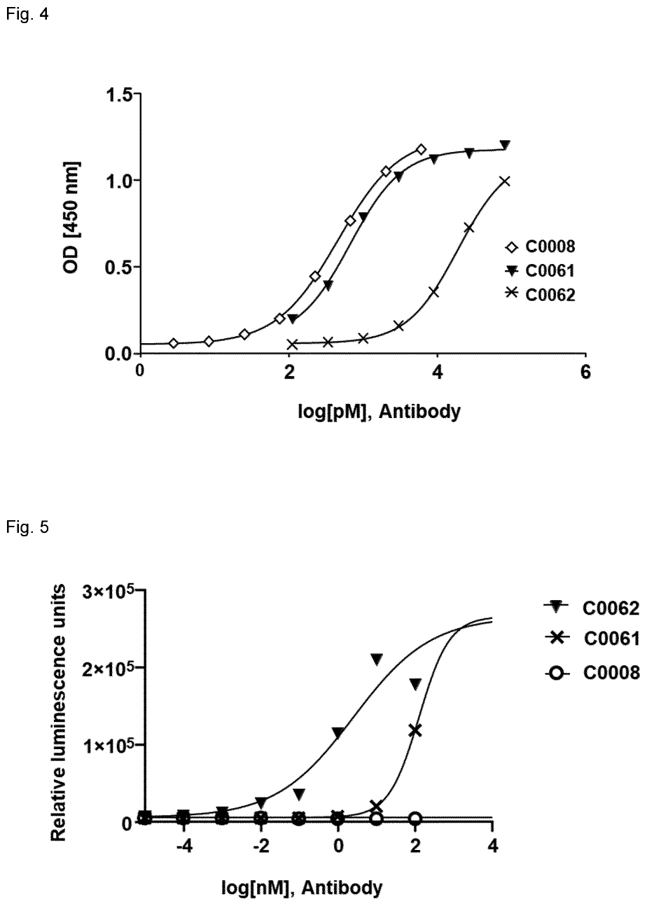

The binding curves of anti-oxMIF/CD3 bispecific antibodies C0061 and C0062 binding towards immobilized MIF (oxMIF) in an ELISA are depicted in FIG. 4. The monospecific anti-oxMIF antibody C0008 was used as positive control for oxMIF binding. EC50 values of the binding curves, which reflect rough KD estimates, were calculated by 4-parameter fit. The experiment was done in triplicate and the mean EC50 values are shown in Table 5.

TABLE 5
EC50 values of bispecific antibodies (ELISA):

| Entity | EC₅₀ (nM) |
|---|---|
| C0061 | 0.9 |
| C0062 | 24.7 |
| C0008 | 0.6 |

### Example 6: Activation of T Cells by C0061 and C0062

The T Cell Activation Bioassay was done according to the Promega technical manual for product J1621 by using genetically engineered Jurkat T cells (effector cells) that express a luciferase reporter driven by a NFAT-response element.

Activation of T cells by anti-oxMIF/CD3 bispecific antibodies C0061 and C0062 is shown in FIG. 5. Anti-oxMIF monospecific antibody C0008 was used as control.

### Example 7: PBMC Mediated Tumor Cell Killing

CFSE (Carboxyfluoresceinsuccinimidylester)-stained HCT116 human colorectal cancer cells were seeded in 96 well flat bottom plates. PBMCs were isolated from blood of healthy, human donors. Serial dilutions of anti-oxMIF/CD3 bispecific antibodies were added to the tumor cells together with PBMCs and incubated for 22 h (Effector-to-target cell ratio: 2.5:1). The medium (containing PBMCs) was removed. Adherent cells were trypsinized, stained with a dead cell staining reagent (Sytox™) and analysed by flow cytometry allowing the calculation of specific killing of stained cancer cells.

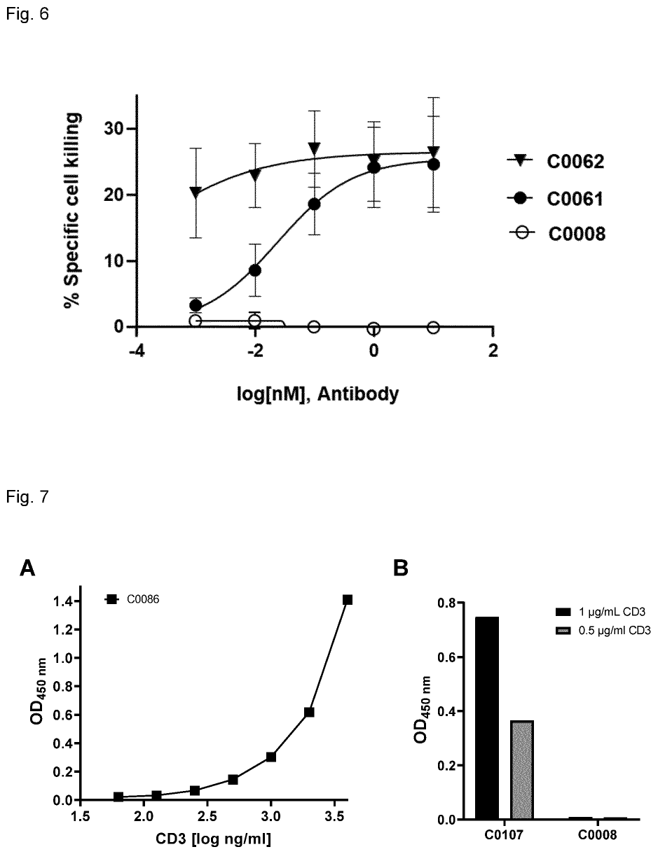

PBMC mediated tumor cell killing of HCT116 human colon cancer cells in the presence of anti-oxMIF/CD3 bispecific constructs C0061 and C0062 is shown in FIG. 6. Anti-oxMIF monospecific antibody C0008 was used as control. The experiment was repeated using PBMCs from 5 different donors and the mean and standard deviation of the specific cell killing (as percentage of total cancer cells) were calculated and plotted against the concentrations of the antibodies.

### Example 8: oxMIF-CD3 Bridging ELISA of C0086 and C0107

Simultaneous binding of bispecific antibodies C0086 and C0107 to oxMIF and CD3 was determined as described in Example 4, with concentrations of FLAG-tagged CD3E/δ-Fc fusion protein as shown in FIG. 7.

Results: It is evident from FIG. 7 that both molecules C0086 and C0107 were able to bind simultaneously to oxMIF and CD3.

FIG. 7 shows simultaneous binding of anti-oxMIF/CD3 bispecific antibodies C0086 and C0107 to oxMIF and CD3. The anti-oxMIF monospecific antibody C0008 was used as negative control.

### Example 9: Binding to Immobilized oxMIF (ELISA)

The binding of anti-oxMIF/CD3 bispecific antibodies C0086 and C0107 towards immobilized MIF (oxMIF) was determined by ELISA as described in Example 5. The monospecific anti-oxMIF antibody C0008 was used as positive control for oxMIF binding, and the signal obtained at the highest concentration of this antibody was set to 100% to normalize datasets from different experiments.

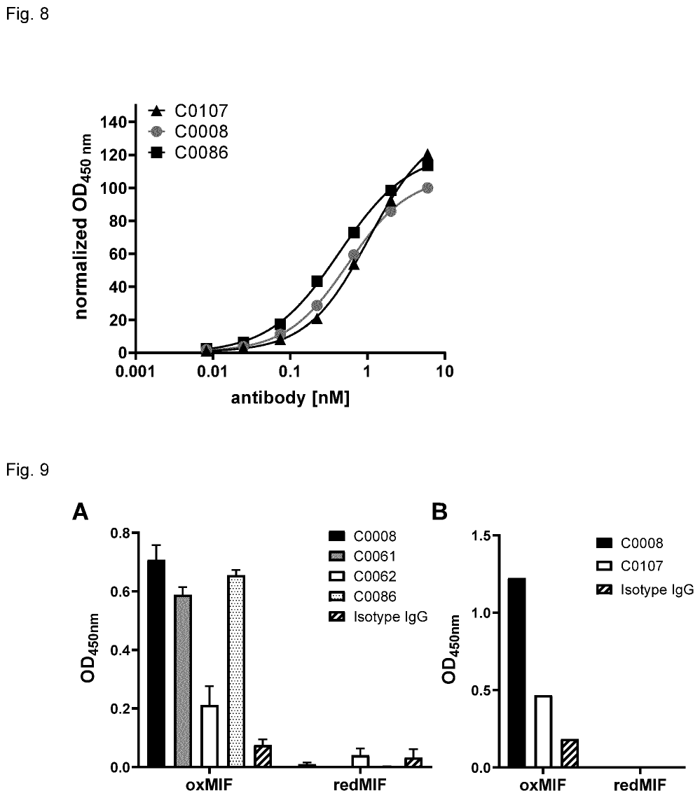

Results: It is evident from FIG. 8 that both anti-oxMIF/CD3 bispecific antibodies C0086 and C0107 showed comparable binding towards oxMIF as similar binding curves were obtained over the whole range of antibody concentrations.

FIG. 8 shows the binding of anti-oxMIF/CD3 bispecific antibodies C0086 and C0107 to immobilized oxMIF in an ELISA. The anti-oxMIF monospecific antibody C0008 was used as positive control.

### Example 10: Differential Binding of Anti-oxMIF/CD3 Bispecific Antibodies C0061, C0062, C0086 and C0107 to oxMIF Vs. redMIF

Antibodies which are bivalent for oxMIF and an isotype control antibody were immobilized into microplates over night at 4° C. at a concentration of 15 nM. Molecules which are monovalent for oxMIF were immobilized at a concentration of 30 nM. After blocking, wells were incubated with 50 ng/ml of either redMIF or the oxMIF surrogate NTB-MIF (Schinagl et al., 2018). Captured oxMIF was either detected with a biotinylated polyclonal rabbit anti-MIF antibody and Streptavidin-HRP conjugate (FIG. 9A) or non-biotinylated polyclonal rabbit anti-MIF antibody and goat anti-rabbit-HRP conjugate (FIG. 9 B). Plates were stained with tetramethylbenzidine (TMB) and chromogenic reaction was stopped by addition of 30% H₂SO₄. OD was measured at 450 nm.

Results: The results clearly showed that the anti-oxMIF/CD3 bispecific antibodies C0061, C0062, C0086 and C0107 bind to oxMIF, but no binding to redMIF was detected. Thus, the antibodies retained their ability to discriminate between oxMIF and redMIF (FIGS. 9A and B). The mean of two or three independent experiments is shown.

FIG. 9 shows the differential binding of the anti-oxMIF/CD3 bispecific antibodies (A) C0061, C0062, C0086 and (B) C0107 to oxMIF vs. redMIF. Imalumab (C0008) was used as reference antibody and a non-specific isoype IgG as negative control.

### Example 11: Binding of Bispecific Antibodies to Native CD3 Expressed on Jurkat T-cells

CD3 positive (CD3+) Jurkat T-cells (ATCC, TIB-152), which express functional CD3 and CD3 negative (CD3-) Jurkat T-cells (ATCC, TIB-153) lacking expression of CD3, were incubated with bispecific antibodies or C0008 (anti-oxMIF monospecific control antibody) at a concentration of 33 nM or with secondary antibody only (control). Bound antibodies were detected by a goat anti-human IgG (H+L) Alexa-Fluor 488 conjugate (secondary antibody). Fixable Viability Dye eFluor™ 780 was used to exclude dead cells and samples were analysed by FACS. Data were analysed using FlowJow software and the mean fluorescence intensity (MFI) of viable stained cells is shown.

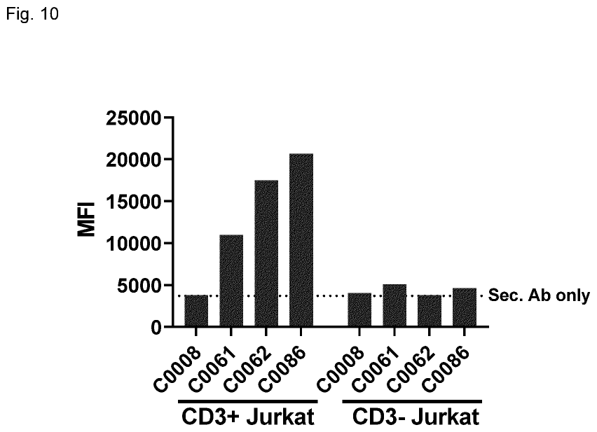

Results: FIG. 10 evidently shows that the anti-oxMIF/CD3 bispecific antibodies specifically bound to native CD3 expressed on viable Jurkat T-cells, whereas only background staining was detected on Jurkat T-cells lacking expression of CD3. Background staining was determined by measuring cells stained with secondary ab only. No binding was further observed with monospecific anti-oxMIF antibody (C0008).

FIG. 10 shows the specific binding of anti-oxMIF/CD3 bispecific antibodies to native CD3 expressed on CD3-positive Jurkat T-cells, whereas only background staining was determined on CD3-negative Jurkat T-cells. The monospecific anti oxMIF antibody C0008 was used as negative control.

### Example 12: IL-2 Secretion of Human T-Cells Activated by an Anti-oxMIF/CD3 Bispecific Antibody in the Presence of Target Cells

Human PBMCs isolated from healthy donors were treated with oxMIF/CD3 bispecific antibody C0061 or monospecific anti-oxMIF antibody C0008 at concentrations ranging from 0.01 nM-10 nM, either in presence or in absence of HCT116 cancer cells (effector to target cell ratio 2.5:1). After 24 hours of incubation at 37° C., supernatants were collected, and interleukin-2 (IL-2) concentrations were assessed using the LEGENDplex bead-based immunoassay (BioLegend).

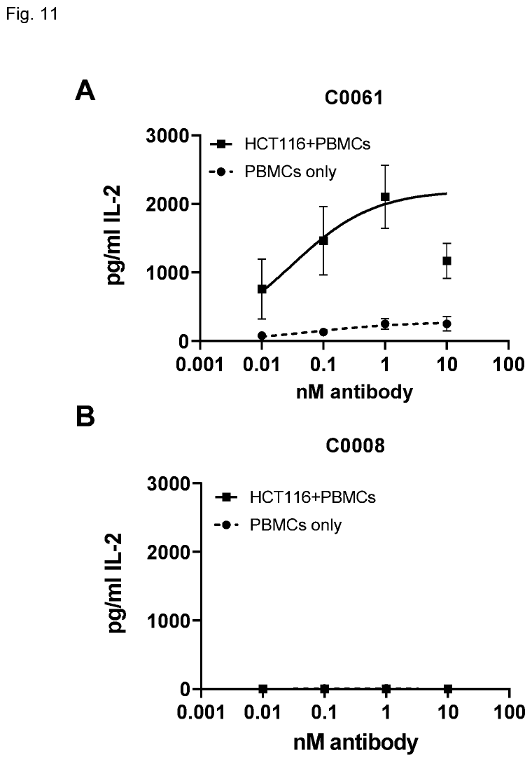

Results: IL-2 is secreted from T-cells indicating T-cell activation upon tumor cell engagement. FIG. 11A demonstrates that T-cells were activated by crosslinking with HCT116 tumor cells through C0061, leading to significant release of IL-2 into the cell culture supernatant. T-cells incubated with C0061 in the absence of cancer cells showed an approximately 10-fold reduced IL-2 secretion. The anti-oxMIF monospecific antibody 00008 which was used as a negative control did not induce any IL-2 secretion from T-cells, neither in absence nor in the presence of cancer cells (FIG. 11 B). Data are shown as mean +/−SEM of four different PBMC donors.

FIG. 11 shows the IL-2 secretion of activated human T cells by anti-oxMIF/CD3 bispecific antibody C0061, either in the presence or in the absence of human HCT116 target cells.

### Example 13: PBMC Mediated Tumor Cell Killing of HCT116 and A2780 Cancer Cells with Increased Cell Surface Presentation of oxMIF

A2780 and HCT116 cells were transfected with the HaloTag-HiBiT plasmid (Promega #CS1956B17), selected with blasticidin and sorted as cell pools. Stable HiBiT-expressing cell lines were then transfected with a MIF-pDisplay plasmid (Invitrogen), selected with geneticin, and sorted to generate cell lines stably expressing intracellular HiBiT and membrane-anchored monomeric oxMIF (termed A2780-HiBiT-pMIF and HCT116-HiBiT-pMIF), i.e. MIF is displayed in a non-native monomeric state which makes the epitope accessible to anti-oxMIF antibodies (Schinagl et al., Biochemistry 2018). These cell lines show increased presentation of oxMIF at the cellular surface and are therefore a more sensitive tool for in vitro analysis.

A2780-HiBiT-pMIF or HCT116-HiBiT-pMIF cells were seeded into 96-well plates and left to adhere overnight. PBMCs isolated from healthy donors (n=3) were added at effector-to-target ratios of 2.5:1 (A2780) or 10:1 (HCT116) in the presence or absence of anti-oxMIF/CD3 bispecific antibodies or monospecific anti-oxMIF antibody C0008 at concentrations ranging from 0.001-100 nM. After 24 hours of incubation, Nano-Glo HiBiT Extracellular Detection Reagent (Promega #N2421) was added, and luminescence signals were measured on a Tecan plate reader.

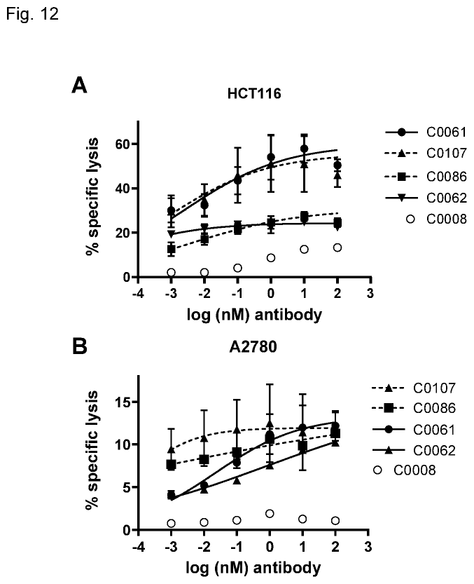

Results: FIG. 12 shows PBMC mediated tumor cell killing induced by the anti-oxMIF/CD3 bispecific antibodies C0061, C0062, C0086 and C0107 using oxMIF displaying human colon cancer cells HCT116 (FIG. 12A) and human ovarian cancer cells A2780 (FIG. 12 B) as target cells. Monospecific anti-oxMIF antibody C0008 was used as control for determining non-specific PBMC mediated cancer cell lysis. PBMC-mediated lysis of cancer cells is presented as mean +/−SEM (as percentage of total cancer cells) and was plotted against the concentrations of the antibodies.

FIG. 12: PBMC mediated tumor cell killing of oxMIF displaying colon cancer cells HCT116 (A) and human ovarian cancer cells A2780 (B) induced by anti-oxMIF/CD3 bispecific antibodies. The anti-oxMIF monospecific antibody C0008 was used as negative control.

### Example 14: Pharmacokinetics (PK) of C0061 in the Circulation of NSG Mice

Pharmacokinetics of C0061 after intravenous injection was investigated in NSG mice. NSG mice received a single intravenous dose of C0061 of either 20, 10 or 3 mg/kg, respectively. After 4, 10, 24, 48 and 72 hours, 20 μl blood was collected by tail vein puncture using K3-EDTA-coated Minivette® and was transferred into K3-EDTA-coated vials containing 60 μl PBS. After centrifugation, the supernatant (=1:4-diluted plasma) was used to determine C0061 concentration by ELISA. Briefly, recombinant human MIF diluted in PBS at 1 μg/ml was immobilized into ELISA plates overnight at 4° C. (transforming MIF to oxMIF according to Thiele et al., 2015). After blocking with 2% fish gelatin/TBST, diluted mouse plasmas (1:100-1:10,000) were added to the plates. The standard curve was obtained by adding a serial dilution of C0061 (0.05-100 ng/ml) to the plate. Finally, bound C0061 was detected using goat anti human Fc-HRP conjugate and tetramethylbenzidine (TMB) as substrate. The chromogenic reaction was stopped with 3 M H2504 and OD was measured at 450 nm. Concentrations of C0061 in mouse plasma were calculated from the C0061 standard curve by non-linear regression using a hyperbola curve fit using GraphPad Prism. The resulting data were fitted to an equation describing a biexponential decay in GraphPad Prism to determine the initial and terminal half-life of C0061 in NSG mice.

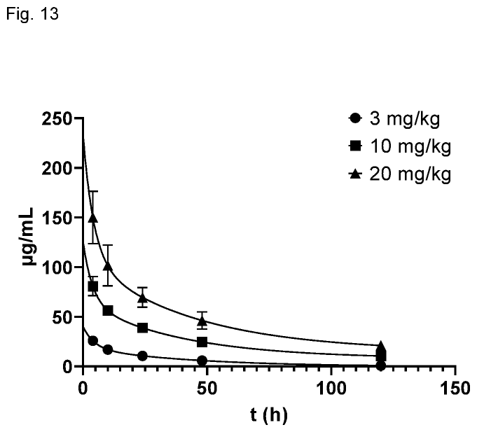

Results: The pharmacokinetic profile of C0061 demonstrated a biexponential decay as expected with an initial half-life of 3 hours and a terminal half-life of 30 hours (FIG. 13). Additionally, the measured plasma concentration of C0061 increased linearly with antibody dose.

FIG. 13 shows the pharmacokinetics (PK) of C0061 in the circulation of NSG mice after intravenous injection.

### Example 15: Biodistribution of Anti-oxMIF/CD3 Bispecific Antibody C0061 in CALU-6 Lung Cancer Bearing NSG Mice

Biodistribution of anti-oxMIF/CD3 bispecific antibody C0061 was investigated in xenograft model of NSG mice carrying subcutaneous tumors of the human lung cancer cell line CALU-6. Female NSG mice received unilateral, subcutaneous injections of 5×10⁶ CALU-6 cells in PBS (100 μl/animal). Upon reaching individual tumor volumes of 150-300 mm³, mice were assigned to treatment groups and received a single intravenous dose of 5 mg/kg IRDye 800CW-labeled C0061. Two untreated mice were used as ‘no signal’ controls.

C0061 was labelled with IRDye 800CW using the IRDye 800CW Protein labelling kit—high MW from LI-COR Biosciences following the manufacturer's instructions. After the labelling process and prior to injection of labelled antibodies into mice, the protein concentration and labelling efficiency of the IRDye 800CW labelled antibody was determined using the Nanodrop technology, and mice were dosed based on the protein concentration after labelling. In vivo imaging was performed in a LI-COR Pearl® Trilogy imaging device upon administration of labelled antibodies at the following time-points: 1 h, 6 h, 24 h, 48 h, 72 h, 96 h, 168 h after dosing.

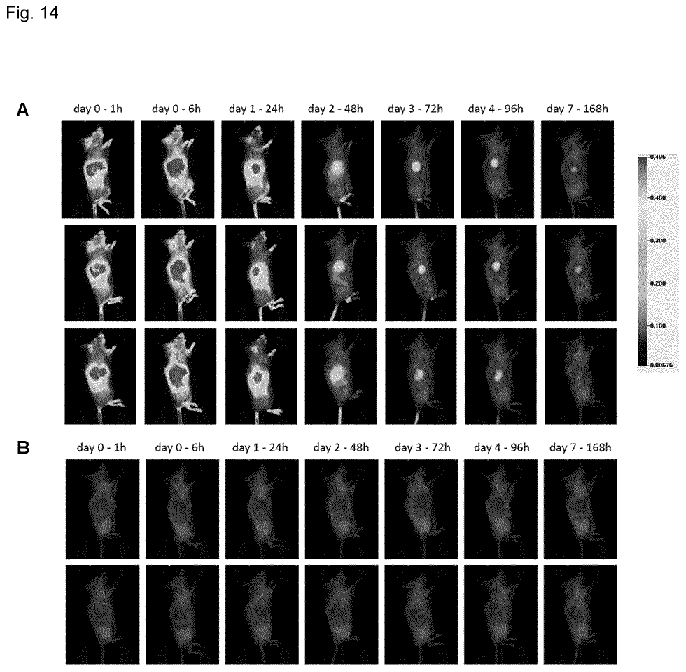

Results: A clear intra-tumoral distribution of intravenously administered 800CW-labeled C0061 (FIG. 14A) was determined, with a peak signal at 24 h and tumor retention of up to 7 days. This clearly demonstrates the accumulation and retention of C0061 in the tumor, which is a prerequisite for recruitment of cytotoxic T-cells to the tumor by its CD3 binding portion. No signal was detected in untreated control mice (FIG. 14 B).

FIG. 14 shows the tumor penetration and accumulation of C0061 by infra-red in vivo imaging of mice carrying subcutaneous CALU-6 tumors. Pictures were taken 1 h, 6 h, 24 h, 48 h, 72 h, 96 h and 168 h post injection of the IRDye 800CW labelled antibody. A: Mice which received IRDye 800CW-labeled C0061 (5 mg/kg); B: non-treated control mice; Scalebar is the same for A and B.

1. An anti-oxMIF/anti-CD3 antibody; selected from the group consisting of:
(i) an IgG wherein a scFv is fused to only one of the two heavy chains;
(ii) an IgG wherein one Fab arm is replaced by a bispecific-T-cell-engager (BiTE) and one Fab arm is an IgG Fab arm and wherein said BiTE and IgG Fab arm are linked to the Fc-portion via the hinge region; and
(iii) an IgG wherein both Fab arms are replaced by scFvs with different specificities,
wherein the antibody comprises at least one binding site specifically recognizing oxMIF and one binding site specifically recognizing CD3,
and wherein the site specifically recognizing oxMIF comprises:
(a) a variable CDR comprising sequences SEQ ID NOs: 1 to 6, or a variable CDR region with at least 70% sequence identity to SEQ ID NOs: 1 to 6, or
(b) a variable CDR comprising sequences SEQ ID NOs: 7 to 12, or a variable CDR with at least 70% sequence identity to SEQ ID NOs: 7 to 12, or
(c) a variable CDR comprising sequences SEQ ID NOs: 13 to 18, or a variable CDR with at least 70% sequence identity to SEQ ID NOs: 13 to 18, or
(d) a variable CDR comprising sequences SEQ ID NOs: 19 to 24, or a variable CDR with at least 70% sequence identity to SEQ ID NOs:19 to 24, or
(e) a variable CDR comprising sequences SEQ ID NOs: 26, 27, 21, 28, 23, and 24, or a variable CDR with at least 70% sequence identity to SEQ ID NOs: 26, 27, 21, 28, 23, or
(f) a variable CDR comprising sequences SEQ ID NOs:19, 20, 21, 138, 25, and 153, or a variable CDR with at least 70% sequence identity to SEQ ID NOs: 19, 20, 21, 138, 25, and 153.

2. The anti-oxMIF/anti-CD3 antibody of claim 1, comprising 0, 1, or 2 point mutations in each of the CDR sequences.

3. The anti-oxMIF/anti-CD3 antibody according to claim 1, wherein the binding site specifically recognizing CD3 comprises a variable region comprising 0, 1, or 2 point mutations in each of the following CDR sequences:
SEQ ID NOs: 77, 78, 149, 83, 84 and 151, or
SEQ ID NOs: 77, 78, 79, 80, 81 and 82, or
SEQ ID NOs: 77, 78, 79, 83, 84 and 85, or
SEQ ID NOs: 77, 154, 79, 83, 84 and 85, or
SEQ ID NOs: 86, 87, 88, 89, 90 and 91, or
SEQ ID NOs: 92, 93, 94, 95, 96 and 97, or
SEQ ID NOs: 167, 168, 169, 178, 179, and 180, or
SEQ ID NOs: 170, 171, 172, 181, 182 and 183.

4. The anti-oxMIF/anti-CD3 antibody according to claim 1, comprising 0 or 1 point mutation in one or more of the following sequences: SEQ ID NOs: 7, 8, 9, 10, 11, 12, 167, 168, 169, 178, 179 and 180.

5. The anti-oxMIF/anti-CD3 antibody according to claim 1, comprising one or more of the following sequences: SEQ ID NOs: 7, 8, 9, 10, 11, 12, 77, 78, 149, 83, 84, and 151.

6. The anti-oxMIF/anti-CD3 antibody according to claim 1, wherein the IgG is recognizing oxMIF and the scFv fused to one of the heavy chains is recognizing CD3, further comprising a peptide linker joining the anti-CD3 variable light (VL) and variable heavy (VH) chains.

7. The anti-oxMIF/anti-CD3 antibody according to claim 1, wherein the IgG Fab arm is recognizing oxMIF and the bispecific T-cell engager (BiTE) is recognizing oxMIF and CD3, further comprising peptide linkers joining the VL and VH chains of the bispecific T-cell engager.

8. The anti-oxMIF/anti-CD3 antibody according to claim 1, wherein both Fab arms are replaced by scFvs and wherein one scFv is recognizing oxMIF and the other scFv is recognizing CD3, further comprising peptide linkers joining the VL and VH chains of the scFvs.

9. The anti-oxMIF/anti-CD3 antibody according to claim 1, wherein the binding site specifically recognizing oxMIF comprises a heavy chain variable region having at least 80%, at least 90%, at least 95%, or at least 99% sequence identity to the amino acid sequence of SEQ ID NO: 158, and a light chain variable region having at least 80%, at least 90%, at least 95%, or at least 99% sequence identity to the amino acid sequence of SEQ ID NO: 134.

10. The anti-oxMIF/anti-CD3 antibody according to claim 1, wherein the binding site specifically recognizing CD3 comprises a heavy chain variable region having at least 80%, at least 90%, or at least 95% sequence identity to the amino acid sequence of SEQ ID NO: 135 and a light chain variable region having at least 80%, at least 90%, or at least 95% sequence identity to the amino acid sequence of SEQ ID NO: 136.

11. The anti-oxMIF/anti-CD3 antibody according to claim 1, comprising the amino acid sequence of SEQ ID NOs: 159, 137, 140, 160, 161, 162, 163, 194, 195, 196, or 197, or an amino acid sequence having at least 85%, 90%, 95%, or 99% sequence identity with any one of SEQ ID NOs: 159, 137, 140, 160, 161, 162, 163, 194, 195, 196, or 197.

12. The anti-oxMIF/anti-CD3 antibody according to claim 1, further comprising a pharmaceutically acceptable carrier or excipient.

13. A method of treating a disease selected from the group consisting of colorectal cancer, ovarian cancer, pancreas cancer, and lung cancer, comprising the step of administering a therapeutically effective amount of the anti-oxMIF/anti-CD3 antibody according to claim 1 to a subject in need thereof.

14. (canceled)

15. Isolated nucleic acid molecule(s) encoding an anti-oxMIF/anti-CD3 antibody according to claim 1.

16. The nucleic acid molecule(s) of claim 15, wherein the nucleic acid molecule(s) are incorporated into an expression vector.

17. The nucleic acid molecule(s) of claim 16, wherein the vector is incorporated into a host cell.

18-22. (canceled)


SEQUENCE LISTING (197 sequences)

SEQ ID NO: 1 - protein, 5 aa, Artificial Sequence
  HV-CDR1 (CDR1-H1)
IYTMD

SEQ ID NO: 2 - protein, 17 aa, Artificial Sequence
  HV-CDR2 (CDR2-H1)
YISPSGGNTSYADSVKG

SEQ ID NO: 3 - protein, 17 aa, Artificial Sequence
  HV-CDR3 (CD3-H1)
RQYVLRYFDWSADAFDI

SEQ ID NO: 4 - protein, 11 aa, Artificial Sequence
  LV-CDR1 (CDR1-L1)
RASQSISSYLN

SEQ ID NO: 5 - protein, 7 aa, Artificial Sequence
  LV-CDR2 (CDR2-L1)
AASSLQS

SEQ ID NO: 6 - protein, 9 aa, Artificial Sequence
  LV-CDR3 (CDR3-L1)
QQSYSTPWT

SEQ ID NO: 7 - protein, 5 aa, Artificial Sequence
  HV-CDR1 (CDR1-H1)
IYSMN

SEQ ID NO: 8 - protein, 17 aa, Artificial Sequence
  HV-CDR2 (CDR2-H1)
SIGSSGGTTYYADSVKG

SEQ ID NO: 9 - protein, 9 aa, Artificial Sequence
  HV-CDR3 (CD3-H1)
SQWLYGMDV

SEQ ID NO: 10 - protein, 11 aa, Artificial Sequence
  LV-CDR1 (CDR1-L1)
RSSQRIMTYLN

SEQ ID NO: 11 - protein, 7 aa, Artificial Sequence
  LV-CDR2 (CDR2-L1)
VASHSQS

SEQ ID NO: 12 - protein, 9 aa, Artificial Sequence
  LV-CDR3 (CDR3-L1)
QQSFWTPLT

SEQ ID NO: 13 - protein, 5 aa, Artificial Sequence
  HV-CDR1 (CDR1-H1)
KYYMI

SEQ ID NO: 14 - protein, 17 aa, Artificial Sequence
  HV-CDR2 (CDR2-H1)
WIGPSGGFTFYADSVKG

SEQ ID NO: 15 - protein, 12 aa, Artificial Sequence
  HV-CDR3 (CD3-H1)
GTPDYGGNSLDH

SEQ ID NO: 16 - protein, 11 aa, Artificial Sequence
  LV-CDR1 (CDR1-L1)
RASQSIGTYLS

SEQ ID NO: 17 - protein, 7 aa, Artificial Sequence
  LV-CDR2 (CDR2-L1)
ATSRLQS

SEQ ID NO: 18 - protein, 9 aa, Artificial Sequence
  LV-CDR3 (CDR3-L1)
QQTYSTPLT

SEQ ID NO: 19 - protein, 5 aa, Artificial Sequence
  HV-CDR1 (CDR1-H1)
IYAMD

SEQ ID NO: 20 - protein, 17 aa, Artificial Sequence
  HV-CDR2 (CDR2-H1)
GIVPSGGFTKYADSVKG

SEQ ID NO: 21 - protein, 18 aa, Artificial Sequence
  HV-CDR3 (CD3-H1)
VNVIAVAGTGYYYYGMDV

SEQ ID NO: 22 - protein, 12 aa, Artificial Sequence
  LV-CDR1 (CDR1-L1)
RASQGVSSSSLA

SEQ ID NO: 23 - protein, 7 aa, Artificial Sequence
  LV-CDR2 (CDR2-L1)
GTSSRAT

SEQ ID NO: 24 - protein, 8 aa, Artificial Sequence
  LV-CDR3 (CDR3-L1)
QQYGRSLT

SEQ ID NO: 25 - protein, 7 aa, Artificial Sequence
  LV-CDR2 (CDR2-L1)
GASNRAT

SEQ ID NO: 26 - protein, 5 aa, Artificial Sequence
  HV-CDR1 (CDR1-H1)
WYAMD

SEQ ID NO: 27 - protein, 17 aa, Artificial Sequence
  HV-CDR2 (CDR2-H1)
GIYPSGGRTKYADSVKG

SEQ ID NO: 28 - protein, 12 aa, Artificial Sequence
  LV-CDR1 (CDR1-L1)
RASQGVSSSSLA

SEQ ID NO: 29 - protein, 30 aa, Artificial Sequence
  HV-FR1
EVQLLESGGGLVQPGGSLRLSCAASGFTFS

SEQ ID NO: 30 - protein, 14 aa, Artificial Sequence
  HV-FR2
WVRQAPGKGLEWVS

SEQ ID NO: 31 - protein, 32 aa, Artificial Sequence
  HV-FR3
RFTISRDNSKNTLYLQMNSLRAEDTAVYYCAS

SEQ ID NO: 32 - protein, 11 aa, Artificial Sequence
  HV-FR4
WGQGTMVTVSS

SEQ ID NO: 33 - protein, 23 aa, Artificial Sequence
  LV-FR1
DIQMTQSPSSLSASVGDRVTITC

SEQ ID NO: 34 - protein, 15 aa, Artificial Sequence
  LV-FR2
WYQQKPGKAPKLLIY

SEQ ID NO: 35 - protein, 32 aa, Artificial Sequence
  LV-FR3
GVPSRFSGSGSGTDFTLTISSLQPEDFATYYC

SEQ ID NO: 36 - protein, 10 aa, Artificial Sequence
  LV-FR4
FGQGTKVEIK

SEQ ID NO: 37 - protein, 30 aa, Artificial Sequence; identical to SEQ ID NO: 29
  HV-FR1

SEQ ID NO: 38 - protein, 14 aa, Artificial Sequence
  HV-FR2
WVRQAPGKGLEWVS

SEQ ID NO: 39 - protein, 32 aa, Artificial Sequence
  HV-FR3
RFTISRDNSKNTLYLQMNSLRAEDTAVYYCAG

SEQ ID NO: 40 - protein, 11 aa, Artificial Sequence
  HV-FR4
WGQGTTVTVSS

SEQ ID NO: 41 - protein, 23 aa, Artificial Sequence
  LV-FR1
DIQMTQSPSSLSASVGDRVTITC

SEQ ID NO: 42 - protein, 15 aa, Artificial Sequence
  LV-FR2
WYQQKPGKAPKLLIF

SEQ ID NO: 43 - protein, 32 aa, Artificial Sequence
  LV-FR3
GVPSRFRGSGSETDFTLTISGLQPEDSATYYC

SEQ ID NO: 44 - protein, 10 aa, Artificial Sequence
  LV-FR4
FGGGTKVEIK

SEQ ID NO: 45 - protein, 30 aa, Artificial Sequence; identical to SEQ ID NO: 29
  HV-FR1

SEQ ID NO: 46 - protein, 14 aa, Artificial Sequence
  HV-FR2
WVRQAPGKGLEWVS

SEQ ID NO: 47 - protein, 32 aa, Artificial Sequence
  HV-FR3
RFTISRDNSKNTLYLQMNSLRAEDTAVYYCAR

SEQ ID NO: 48 - protein, 11 aa, Artificial Sequence
  HV-FR4
WGQGTLVTVSS

SEQ ID NO: 49 - protein, 23 aa, Artificial Sequence
  LV-FR1
DIQMTQSPSSLPASVGDRVTITC

SEQ ID NO: 50 - protein, 15 aa, Artificial Sequence
  LV-FR2
WYQHKPGNAPKLLIY

SEQ ID NO: 51 - protein, 32 aa, Artificial Sequence
  LV-FR3
GVPSRFSGGGSGTRFTLAISSLQPDDFATYFC

SEQ ID NO: 52 - protein, 10 aa, Artificial Sequence
  LV-FR4
FGGGTKVDIK

SEQ ID NO: 53 - protein, 30 aa, Artificial Sequence; identical to SEQ ID NO: 29
  HV-FR1

SEQ ID NO: 54 - protein, 14 aa, Artificial Sequence
  HV-FR2
WVRQAPGKGLEWVS

SEQ ID NO: 55 - protein, 32 aa, Artificial Sequence; identical to SEQ ID NO: 47
  HV-FR3

SEQ ID NO: 56 - protein, 11 aa, Artificial Sequence
  HV-FR4
WGQGTTVTVSS

SEQ ID NO: 57 - protein, 23 aa, Artificial Sequence
  LV-FR1
DIQMTQSPGTLSLSPGERATLSC

SEQ ID NO: 58 - protein, 15 aa, Artificial Sequence
  LV-FR2
WYQQKPGQAPRLLIY

SEQ ID NO: 59 - protein, 32 aa, Artificial Sequence
  LV-FR3
GIPDRFSGSASGTDFTLTISRLQPEDFAVYYC

SEQ ID NO: 60 - protein, 10 aa, Artificial Sequence
  LV-FR4
FGGGTKVEIK

SEQ ID NO: 61 - protein, 30 aa, Artificial Sequence; identical to SEQ ID NO: 29
  HV-FR1

SEQ ID NO: 62 - protein, 14 aa, Artificial Sequence
  HV-FR2
WVRQAPGKGLEWVS

SEQ ID NO: 63 - protein, 32 aa, Artificial Sequence; identical to SEQ ID NO: 47
  HV-FR3

SEQ ID NO: 64 - protein, 11 aa, Artificial Sequence
  HV-FR4
WGQGTTVTVSS

SEQ ID NO: 65 - protein, 23 aa, Artificial Sequence
  LV-FR1
DIQMTQSPVTLSLSPGERATLSC

SEQ ID NO: 66 - protein, 15 aa, Artificial Sequence
  LV-FR2
WYQQKPGQTPRLLIY

SEQ ID NO: 67 - protein, 32 aa, Artificial Sequence
  LV-FR3
GIPDRFSGSGSGTDFTLTISRLEPEDFAVYYC

SEQ ID NO: 68 - protein, 10 aa, Artificial Sequence
  LV-FR4
FGGGTKVEIK

SEQ ID NO: 69 - protein, 30 aa, Artificial Sequence; identical to SEQ ID NO: 29
  HV-FR1

SEQ ID NO: 70 - protein, 14 aa, Artificial Sequence
  HV-FR2
WVRQAPGKGLEWVS

SEQ ID NO: 71 - protein, 32 aa, Artificial Sequence; identical to SEQ ID NO: 47
  HV-FR3

SEQ ID NO: 72 - protein, 11 aa, Artificial Sequence
  HV-FR4
WGQGTTVTVSS

SEQ ID NO: 73 - protein, 23 aa, Artificial Sequence
  LV-FR1
DIQMTQSPGTLSLSPGERATLSC

SEQ ID NO: 74 - protein, 15 aa, Artificial Sequence
  LV-FR2
WYQQKPGQAPRLLIY

SEQ ID NO: 75 - protein, 32 aa, Artificial Sequence; identical to SEQ ID NO: 59
  LV-FR3

SEQ ID NO: 76 - protein, 10 aa, Artificial Sequence
  LV-FR4
FGGGTKVEIK

SEQ ID NO: 77 - protein, 5 aa, Artificial Sequence
  HV-CDR1 (CDR1-H2)
RYTMH

SEQ ID NO: 78 - protein, 17 aa, Artificial Sequence
  HV-CDR2 (CDR2-H2)
YINPSRGYTNYNQKFKD

SEQ ID NO: 79 - protein, 10 aa, Artificial Sequence
  HV-CDR3 (CDR3-H2)
YYDDHYCLDY

SEQ ID NO: 80 - protein, 10 aa, Artificial Sequence
  LV-CDR1 (CDR1-L2)
RASSSVSYMN

SEQ ID NO: 81 - protein, 7 aa, Artificial Sequence
  LV-CDR2 (CDR2-L2)
DTSKVAS

SEQ ID NO: 82 - protein, 9 aa, Artificial Sequence
  LV-CDR3 (CDR3-L2)
QQWSSNPLT

SEQ ID NO: 83 - protein, 10 aa, Artificial Sequence
  LV-CDR1 (CDR1-L2)
SASSSVSYMN

SEQ ID NO: 84 - protein, 7 aa, Artificial Sequence
  LV-CDR2 (CDR2-L2)
DTSKLAS

SEQ ID NO: 85 - protein, 9 aa, Artificial Sequence
  LV-CDR3 (CDR3-L2)
QQWSSNPFT

SEQ ID NO: 86 - protein, 5 aa, Artificial Sequence
  HV-CDR1 (CDR1-H2)
GYGMH

SEQ ID NO: 87 - protein, 17 aa, Artificial Sequence
  HV-CDR2 (CDR2-H2)
VIWYDGSKKYYVDSVKG

SEQ ID NO: 88 - protein, 9 aa, Artificial Sequence
  HV-CDR3 (CDR3-H2)
QMGYWHFDL

SEQ ID NO: 89 - protein, 11 aa, Artificial Sequence
  LV-CDR1 (CDR1-L2)
RASQSVSSYLA

SEQ ID NO: 90 - protein, 7 aa, Artificial Sequence
  LV-CDR2 (CDR2-L2)
DASNRAT

SEQ ID NO: 91 - protein, 10 aa, Artificial Sequence
  LV-CDR3 (CDR3-L2)
QQRSNWPPLT

SEQ ID NO: 92 - protein, 5 aa, Artificial Sequence
  HV-CDR1 (CDR1-H2)
SFPMA

SEQ ID NO: 93 - protein, 17 aa, Artificial Sequence
  HV-CDR2 (CDR2-H2)
TISTSGGRTYYRDSVKG

SEQ ID NO: 94 - protein, 10 aa, Artificial Sequence
  HV-CDR3 (CDR3-H2)
FRQYSGGFDY

SEQ ID NO: 95 - protein, 13 aa, Artificial Sequence
  LV-CDR1 (CDR1-L2)
TLSSGNIENNYVH

SEQ ID NO: 96 - protein, 7 aa, Artificial Sequence
  LV-CDR2 (CDR2-L2)
DDDKRPD

SEQ ID NO: 97 - protein, 9 aa, Artificial Sequence
  LV-CDR3 (CDR3-L2)
HSYVSSFNV

SEQ ID NO: 98 - protein, 30 aa, Artificial Sequence
  HV-FR1
DIKLQQSGAELARPGASVKMSCKTSGYTFT

SEQ ID NO: 99 - protein, 14 aa, Artificial Sequence
  HV-FR2
WVKQRPGQGLEWIG

SEQ ID NO: 100 - protein, 32 aa, Artificial Sequence
  HV-FR3
KATLTTDKSSSTAYMQLSSLTSEDSAVYYCAR

SEQ ID NO: 101 - protein, 11 aa, Artificial Sequence
  HV-FR4
WGQGTTLTVSS

SEQ ID NO: 102 - protein, 23 aa, Artificial Sequence
  LV-FR1
DIQLTQSPAIMSASPGEKVTMTC

SEQ ID NO: 103 - protein, 15 aa, Artificial Sequence
  LV-FR2
WYQQKSGTSPKRWIY

SEQ ID NO: 104 - protein, 32 aa, Artificial Sequence
  LV-FR3
GVPYRFSGSGSGTSYSLTISSMEAEDAATYYC

SEQ ID NO: 105 - protein, 10 aa, Artificial Sequence
  LV-FR4
FGAGTKLELK

SEQ ID NO: 106 - protein, 30 aa, Artificial Sequence
  HV-FR1
QVQLQQSGAELARPGASVKMSCKASGYTFT

SEQ ID NO: 107 - protein, 23 aa, Artificial Sequence
  LV-FR1
QIVLTQSPAIMSASPGEKVTMTC

SEQ ID NO: 108 - protein, 32 aa, Artificial Sequence
  LV-FR3
GVPAHFRGSGSGTSYSLTISGMEAEDAATYYC

SEQ ID NO: 109 - protein, 10 aa, Artificial Sequence
  LV-FR4
FGSGTKLEIN

SEQ ID NO: 110 - protein, 30 aa, Artificial Sequence
  HV-FR1
QVQLVQSGGGVVQPGRSLRLSCKASGYTFT

SEQ ID NO: 111 - protein, 14 aa, Artificial Sequence
  HV-FR2
WVRQAPGKGLEWIG

SEQ ID NO: 112 - protein, 32 aa, Artificial Sequence
  HV-FR3
RFTISRDNSKNTAFLQMDSLRPEDTGVYFCAR

SEQ ID NO: 113 - protein, 11 aa, Artificial Sequence
  HV-FR4
WGQGTPVTVSS

SEQ ID NO: 114 - protein, 23 aa, Artificial Sequence
  LV-FR1
DIQMTQSPSSLSASVGDRVTITC

SEQ ID NO: 115 - protein, 15 aa, Artificial Sequence
  LV-FR2
WYQQTPGKAPKRWIY

SEQ ID NO: 116 - protein, 32 aa, Artificial Sequence
  LV-FR3
GVPSRFSGSGSGTDYTFTISSLQPEDIATYYC

SEQ ID NO: 117 - protein, 10 aa, Artificial Sequence
  LV-FR4
FGQGTKLQIT

SEQ ID NO: 118 - protein, 30 aa, Artificial Sequence
  HV-FR1
QVQLVESGGGVVQPGRSLRLSCAASGFKFS

SEQ ID NO: 119 - protein, 14 aa, Artificial Sequence
  HV-FR2
WVRQAPGKGLEWVA

SEQ ID NO: 120 - protein, 32 aa, Artificial Sequence; identical to SEQ ID NO: 47
  HV-FR3

SEQ ID NO: 121 - protein, 11 aa, Artificial Sequence
  HV-FR4
WGRGTLVTVSS

SEQ ID NO: 122 - protein, 23 aa, Artificial Sequence
  LV-FR1
EIVLTQSPATLSLSPGERATLSC

SEQ ID NO: 123 - protein, 15 aa, Artificial Sequence
  LV-FR2
WYQQKPGQAPRLLIY

SEQ ID NO: 124 - protein, 32 aa, Artificial Sequence
  LV-FR3
GIPARFSGSGSGTDFTLTISSLEPEDFAVYYC

SEQ ID NO: 125 - protein, 10 aa, Artificial Sequence
  LV-FR4
FGGGTKVEIK

SEQ ID NO: 126 - protein, 30 aa, Artificial Sequence; identical to SEQ ID NO: 29
  HV-FR1

SEQ ID NO: 127 - protein, 14 aa, Artificial Sequence
  HV-FR2
WVRQAPGKGLEWVS

SEQ ID NO: 128 - protein, 32 aa, Artificial Sequence
  HV-FR3
RFTISRDNSKNTLYLQMNSLRAEDTAVYYCAK

SEQ ID NO: 129 - protein, 11 aa, Artificial Sequence
  HV-FR4
WGQGTLVTVSS

SEQ ID NO: 130 - protein, 22 aa, Artificial Sequence
  LV-FR1
DIQLTQPNSVSTSLGSTVKLSC

SEQ ID NO: 131 - protein, 15 aa, Artificial Sequence
  LV-FR2
WYQLYEGRSPTTMIY

SEQ ID NO: 132 - protein, 34 aa, Artificial Sequence
  LV-FR3
GVPDRFSGSIDRSSNSAFLTIHNVAIEDEAIYFC

SEQ ID NO: 133 - protein, 10 aa, Artificial Sequence
  LV-FR4
FGGGTKLTVL

SEQ ID NO: 134 - protein, 107 aa, Artificial Sequence
  VL anti-oxMIF antibody
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIK

SEQ ID NO: 135 - protein, 119 aa, Artificial Sequence
  HV anti-CD3 antibody
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSS

SEQ ID NO: 136 - protein, 106 aa, Artificial Sequence
  VL anti-CD3 antibody
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 137 - protein, 717 aa, Artificial Sequence
  polypeptide 2 of C0061
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGGGSGGGGSGGGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 138 - protein, 12 aa, Artificial Sequence
  LV-CDR1 (CDR1-L1)
RASQSVRSSYLA

SEQ ID NO: 139 - protein, 703 aa, Artificial Sequence
  anti-oxMIF heavy chain - anti CD3 scFv fusia
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 140 - protein, 214 aa, Artificial Sequence
  anti-oxMIF light chain
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 207 aa, Artificial Sequence
  CD3
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 142 - protein, 171 aa, Artificial Sequence
  CD3
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 143 - protein, 182 aa, Artificial Sequence
  CD3
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 144 - protein, 115 aa, Artificial Sequence
  oxMIF
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 145 - protein, 7 aa, Artificial Sequence
  oxMIF
EPCALCS

SEQ ID NO: 146 - protein, 30 aa, Artificial Sequence
  HV-FR1
QVQLVQSGAEVKKPGASVKVSCKASGYTFT

SEQ ID NO: 147 - protein, 14 aa, Artificial Sequence
  HV-FR2
WVRQAPGQGLEWMG

SEQ ID NO: 148 - protein, 32 aa, Artificial Sequence
  HV-FR3
RVTLTTDKSSSTAYMELSSLRSEDTAVYYCAR

SEQ ID NO: 149 - protein, 10 aa, Artificial Sequence
  HV-CDR3 (CDR3-H2)
YYDDHYSLDY

SEQ ID NO: 150 - protein, 15 aa, Artificial Sequence
  LV-FR2
WYQQKPGKAPKRLIY

SEQ ID NO: 151 - protein, 7 aa, Artificial Sequence
  LV-CDR3 (CDR3-L2)
QQWSSNP

SEQ ID NO: 152 - protein, 12 aa, Artificial Sequence
  LV-FR4
FTFGQGTKLEIK

SEQ ID NO: 153 - protein, 8 aa, Artificial Sequence
  LV-CDR3 (CDR3-L1)
QQYGNSLT

SEQ ID NO: 154 - protein, 17 aa, Artificial Sequence
  HV-CDR2 (CDR2-H2)
YINPSRGYTNYNQKVKD

SEQ ID NO: 155 - protein, 11 aa, Artificial Sequence
  HV-FR4
WGQGTLVTVSS

SEQ ID NO: 156 - protein, 472 aa, Artificial Sequence
  Polypeptide 1 of C0062 without Strep tag
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGGGSGGGGSGGGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 472 aa, Artificial Sequence
  Polypeptide 2 of C0062 without His tag
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 158 - protein, 118 aa, Artificial Sequence
  VH anti-oxMIF antibody
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSS

SEQ ID NO: 159 - protein, 448 aa, Artificial Sequence
  Polypeptide 1 of C0061
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 483 aa, Artificial Sequence
  Polypeptide 1 of C0062
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGGGSGGGGSGGGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKASAWSHPQFEK

SEQ ID NO: 161 - protein, 481 aa, Artificial Sequence
  Polypeptide 2 of C0062
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKAAAHHHHHH

SEQ ID NO: 162 - protein, 703 aa, Artificial Sequence; identical to SEQ ID NO: 139
  Polypeptide 1 of C0086

SEQ ID NO: 163 - protein, 448 aa, Artificial Sequence; identical to SEQ ID NO: 159
  Polypeptide 2 of C0086

SEQ ID NO: 164 - protein, 5 aa, Artificial Sequence
  linker
GGGGS

SEQ ID NO: 165 - protein, 6 aa, Artificial Sequence
  linker
GGSGGS

SEQ ID NO: 166 - protein, 4 aa, Artificial Sequence
  linker
GGGS

SEQ ID NO: 167 - protein, 10 aa, Artificial Sequence
  HV-CDR1
GFTFNKYAMN

SEQ ID NO: 168 - protein, 20 aa, Artificial Sequence
  HD-CDR2
RIRSKYNNYATYYADSVKDS

SEQ ID NO: 169 - protein, 16 aa, Artificial Sequence
  HV-CDR3
VRHGNFGNSYISYWAY

SEQ ID NO: 170 - protein, 10 aa, Artificial Sequence
  HV-CDR1
GFTFSTYAMN

SEQ ID NO: 171 - protein, 19 aa, Artificial Sequence
  HV-CDR2
RIRSKYNNYATYYADSVKG

SEQ ID NO: 172 - protein, 16 aa, Artificial Sequence
  HV-CDR3
VRHGNFGNSYVSWFAY

SEQ ID NO: 173 - protein, 25 aa, Artificial Sequence
  HV-FR1
EVQLVESGGGLVQPGGSLKLSCAAS

SEQ ID NO: 174 - protein, 30 aa, Artificial Sequence
  HV-FR3
RFTISRDDSKNTAYLQMNNLKTEDTAVYYC

SEQ ID NO: 175 - protein, 25 aa, Artificial Sequence
  HV-FR1
EVQLLESGGGLVQPGGSLRLSCAAS

SEQ ID NO: 176 - protein, 14 aa, Artificial Sequence
  HV-FR2
WVRQAPGKGLEWVS

SEQ ID NO: 177 - protein, 30 aa, Artificial Sequence
  HV-FR3
RFTISRDDSKNTLYLQMNSLRAEDTAVYYC

SEQ ID NO: 178 - protein, 14 aa, Artificial Sequence
  LV-CDR1
GSSTGAVTSGNYPN

SEQ ID NO: 179 - protein, 7 aa, Artificial Sequence
  LV-CDR2
GTKFLAP

SEQ ID NO: 180 - protein, 9 aa, Artificial Sequence
  LV-CDR3
VLWYSNRWV

SEQ ID NO: 181 - protein, 14 aa, Artificial Sequence
  LV-CDR1
GSSTGAVTTSNYAN

SEQ ID NO: 182 - protein, 7 aa, Artificial Sequence
  LV-CDR2
GTNKRAP

SEQ ID NO: 183 - protein, 9 aa, Artificial Sequence
  LV-CDR3
ALWYSNLWV

SEQ ID NO: 184 - protein, 22 aa, Artificial Sequence
  LV-FR1
QTVVTQEPSLTVSPGGTVTLTC

SEQ ID NO: 185 - protein, 15 aa, Artificial Sequence
  LV-FR2
WVQQKPGQAPRGLIG

SEQ ID NO: 186 - protein, 32 aa, Artificial Sequence
  LV-FR3
GTPARFSGSLLGGKAALTLSGVQPEDEAEYYC

SEQ ID NO: 187 - protein, 22 aa, Artificial Sequence
  LV-FR1
QAVVTQEPSLTVSPGGTVTLTC

SEQ ID NO: 188 - protein, 15 aa, Artificial Sequence
  LV-FR2
WVQEKPGQAFRGLIG

SEQ ID NO: 189 - protein, 32 aa, Artificial Sequence
  LV-FR3
GTPARFSGSLLGGKAALTLSGAQPEDEAEYYC

SEQ ID NO: 190 - protein, 125 aa, Artificial Sequence
  variable heavy chain CD3
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 191 - protein, 109 aa, Artificial Sequence
  variable light chain CD3
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 192 - protein, 125 aa, Artificial Sequence
  variable heavy chain CD3
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 193 - protein, 109 aa, Artificial Sequence
  variable light chain CD3
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 194 - protein, 456 aa, Artificial Sequence
  C0107 polypeptide 1
EVQLLESGGGLVQPGGSLRLSCAASGFTFSWYAMDWVRQAPGKGLEWVSGIYPSGGRTKYADSVKGRFTISRDNSKNTLYLQMNSLRAEDTAVYYCARVNVIAVAGTGYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPK

SEQ ID NO: 195 - protein, 214 aa, Artificial Sequence
  C0107 polypeptide 3
EIVLTQSPGTLSLSPGERATLSCRASQGVSSSSLAWYQQKPGQAPRLLIYGTSSRATGIPDRFSGSGSGTDFTLTISRLEPEDFAVYYCQQYGRSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 448 aa, Artificial Sequence
  C0111 polypeptide 1
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 727 aa, Artificial Sequence
  C0111 polypeptide 2
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSGGGGSGGGGSDIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK